SEQUENCE LISTING (17 sequences)

SEQ ID NO: 1 - DNA, 4571 bp, Hyoscyamus muticus
  CDS (133)..(4407)
atataactaacttcaccttctattcattcattatcaacaaaataatccattttttatcaaaacttgaaggtgttgttacaagacacaactaatattaattgctgcattttaatttaatcttgttgttccaacatggagccatcagatttaagtaatttccgaggtcgaagtatgagaggaagtatgagaggaagtgtaagggaaaatagtaactcaatatggaggaacaatggagttgaaatattttcaagatcaactagagatgaagatgatgaagaggcattaaaatgggcagcacttgagaaattaccaacatatgatagattaagaaaaggtatattgtttggatcacaaggtactggtgttgctgaagttgatgtagatgatcttggtgttcaacaaaggaagaatttgcttgacagacttgttaaaattgctgaagaagataatgagaagttcttgttgaaactcaagaacaggattgacagggttgggattgattttccatctatagaagtgagatttgagcatctgaatattgaggcagatgcatatgttggtagcagagctttgcctacatttaccaacttcatttctaacttcattgagtccctgctggattcacttcacatccttccatcgaaaaaacgttcagttacaattctcaaggatgttagtggtatcgtcaagccctgtcgaatgactctgcttttaggacctccaggttctgggaaaacaactttgttacttgctttggctggaaaacttgattctgctctaagggttacggggaaggtgacgtataatggacacgaattacatgaatttgtgccacaaagaactgcggcctatattagccagcatgatttgcatattggagaaatgactgtcagagaaactttggagttctctgcaagatgccaaggagttggttctcgttacgaaatgttggccgaactgtcaagaagagagaaagcggctaatatcaaaccagatgctgatattgacatgttcatgaaggctgcatcaactgaagggcaagaagccaaagtgattactgattatgttcttaagattctgggactggatatttgtgcagatactatggtgggagatcaaatgataaggggtatttcaggaggacagaagaagcgtgtcactactggtgaaatgattgtcggaccgtctaaagcccttttcatggatgaaatttcaactggacttgacagttccacaacttactccatcgtgaattccctaaagcaatctgttcaaatcttgaaaggaacagctctgatttctctcttgcagcctgcccccgagacttacaacttgttcgatgatattgttctgctatcagatggctacattgtttatcagggtccacgagaggaagtgctcgatttctttgaatccatgggattcaaatgccccaacagaaaaggcgtggctgacttcttgcaagaagttacatctaagaaggatcaacagcaatattgggtaaagagggacgagccttataggtttattacatcaaaagaatttgctgaggcttatcaatctttccatgttgggagaaaagtaagcgatgaacttacaaccgcatttgacaagagcaaaagccaccctgctgctttgactactgaaaagtatggtattggagtgaaacaacttttgaaggtttgcacggaaagagagttccttctaatgcagaggaattcatttgtttacatcttcaaattctttcagcttatggtaattgcacttatgacaatgaccatattttttcgaactaagatgtctcgggatactgagaccgatggaggaatttattctggtgctctcttttttacggttgttatgcttatgtttaatggtttgtctgagcttcctttgacactctacaagctcccggtcttctacaagcaaagggactttctcttctatccttcatgggcttatgcagttccttcatggatcctaaaaatccctgtaacttttcttgaagttgggatgtgggtgtttctcacctattatgtcatcggatttgatcctaatgttggaagatttttcaaacaatttttgctactcatagtagtaaaccagatggcatcaggattgttcaggtttattgcagcagttggaaggaccatgggagttgctagcacatttggagcatttgcgctgcttttacaatttgcattgggcggttttgtccttgcacgaactgacgtgaaggactggtggatttggggatactggacctcaccacttatgttctcagtgaatgcaatccttgtgaatgaatttgacggaaaaaagtggaaacatattgcgccaaatggaactgagccgcttggacctgcagtggtaagatctcaagggttctttcccgatgcatattggtactggataggtgtaggtgcacttgttggattcacagttctgtttaacatagcctacagtcttgctctcgcttatcttaacccattcggaaagccacaagctacaatttcagaagaaagtgagagcaacgaaaatagtgaattatcaaccccaatagctagtacaacggaaggagattctgtcggtgagaatcagaataagaaaggaatggttcttccatttgaaccccattccatcacctttgatgaagttgtatactcagttgacatgcctccggaaatgagagagcaaggtaccagtgacaatagattggtacttttgaagagtgtgagtggagctttcaggccaggtgttctcacagctctgatgggagttagtggagccggtaaaacaacattgatggatgtcttagctggaaggaaaactggaggttacattgacggaagcattaacatttctggatatcccaagaagcaagaaacatttgcacgtatttctggatactgtgaacaaaacgacatccattcaccttatgtaacagtttatgagtccttggtttactcggcttggctgcgtttacctcaagacgttgatgagaaaaagcgaatgatgttcgttgaacaagttatggaacttgtggagcttacaccactaagatctgccttagtcgggttgccaggagttaatggtctgacgattgcagttgaactagtagcaaacccctctatcatttttatggacgaaccaacttcaggattggatgcaagagctgctgcaattgtgatgagagctgttaggaacactgtcgatacagggagaactgttgtttgtaccattcatcagcctagcattgacatttttgaggcgttcgatgagttatttcttatgaaacgaggaggacaagagatatacgtcggtccattaggtcgtgagtcaagccatttgataaagtattttgagtctatacccggtgtaaccaaaataaaggaggggtacaatccagcaacttggatgttagaagtcacatcttcgtctcaagaaataacattaggtgttgattttaccgaattatacaagaactcagacctcttccggaggaacaaagctttgatcgaggaactaagtgtgccacgccctggtacaagtgacctgcattttgaaactgaattctcacagccattttgggtccaatgtatggcttgtttgtggaagcaacactggtcatactggcgtaatccggcttatactgcagtcagatttctcttcacaaccttcatagctctcatattcgggtcaatgttctgggatattggtacaaaagtgagtgggccccaagatctgaaaaacgccatgggatctatgtatgctgctgtcctcttccttggtgtgcagaattcatcgtcagttcagcccgttgtatctgtcgaacgtactgtattttacagagaaaaagctgctggaatgtactccgcgatgccctatgcctttgcacaagttttcatcgaaattccttatgtatttgtacaagctgttgtctatggtctcattgtctattctatgattggatttgaatggactgctgcaaaattcttttggtacttcttcttcatgttcttcaccttcctctacttcaccttctttggcatgatgaccgtggctgttaccccgaaccaaaatgttgcttcaatcgttgccggattcttctatacagtatggaatctcttctcaggattcatcgttccacgacctcgtattccgatatggtggagatggtactactgggcttgccctgttgcatggacattgtatggtttggttgcatctcaatttggagacctccaagatacaattaatgatcaaactgtggaagatttcttgagaagtagctatggatttaagcatgattttctaggagttgttgcagctgtgatcgttgcatttgcagttgttttcgccttcacatttgctttgggtatcaaggcattcaatttccagagaagatagaaatagtatttatttgtattcccagttgttcatatattcttgaataagcttatgaagttttaagttactgaatatgttatgtcttactaatctttctcaattcccagttttgttgtataataacatgtaataattgttattcaaaaaaaaaaaaaaaaaaa

SEQ ID NO: 2 - protein, 1425 aa, Hyoscyamus muticus
MEPSDLSNFRGRSMRGSMRGSVRENSNSIWRNNGVEIFSRSTRDEDDEEALKWAALEKLPTYDRLRKGILFGSQGTGVAEVDVDDLGVQQRKNLLDRLVKIAEEDNEKFLLKLKNRIDRVGIDFPSIEVRFEHLNIEADAYVGSRALPTFTNFISNFIESLLDSLHILPSKKRSVTILKDVSGIVKPCRMTLLLGPPGSGKTTLLLALAGKLDSALRVTGKVTYNGHELHEFVPQRTAAYISQHDLHIGEMTVRETLEFSARCQGVGSRYEMLAELSRREKAANIKPDADIDMFMKAASTEGQEAKVITDYVLKILGLDICADTMVGDQMIRGISGGQKKRVTTGEMIVGPSKALFMDEISTGLDSSTTYSIVNSLKQSVQILKGTALISLLQPAPETYNLFDDIVLLSDGYIVYQGPREEVLDFFESMGFKCPNRKGVADFLQEVTSKKDQQQYWVKRDEPYRFITSKEFAEAYQSFHVGRKVSDELTTAFDKSKSHPAALTTEKYGIGVKQLLKVCTEREFLLMQRNSFVYIFKFFQLMVIALMTMTIFFRTKMSRDTETDGGIYSGALFFTVVMLMFNGLSELPLTLYKLPVFYKQRDFLFYPSWAYAVPSWILKIPVTFLEVGMWVFLTYYVIGFDPNVGRFFKQFLLLIVVNQMASGLFRFIAAVGRTMGVASTFGAFALLLQFALGGFVLARTDVKDWWIWGYWTSPLMFSVNAILVNEFDGKKWKHIAPNGTEPLGPAVVRSQGFFPDAYWYWIGVGALVGFTVLFNIAYSLALAYLNPFGKPQATISEESESNENSELSTPIASTTEGDSVGENQNKKGMVLPFEPHSITFDEVVYSVDMPPEMREQGTSDNRLVLLKSVSGAFRPGVLTALMGVSGAGKTTLMDVLAGRKTGGYIDGSINISGYPKKQETFARISGYCEQNDIHSPYVTVYESLVYSAWLRLPQDVDEKKRMMFVEQVMELVELTPLRSALVGLPGVNGLTIAVELVANPSIIFMDEPTSGLDARAAAIVMRAVRNTVDTGRTVVCTIHQPSIDIFEAFDELFLMKRGGQEIYVGPLGRESSHLIKYFESIPGVTKIKEGYNPATWMLEVTSSSQEITLGVDFTELYKNSDLFRRNKALIEELSVPRPGTSDLHFETEFSQPFWVQCMACLWKQHWSYWRNPAYTAVRFLFTTFIALIFGSMFWDIGTKVSGPQDLKNAMGSMYAAVLFLGVQNSSSVQPVVSVERTVFYREKAAGMYSAMPYAFAQVFIEIPYVFVQAVVYGLIVYSMIGFEWTAAKFFWYFFFMFFTFLYFTFFGMMTVAVTPNQNVASIVAGFFYTVWNLFSGFIVPRPRIPIWWRWYYWACPVAWTLYGLVASQFGDLQDTINDQTVEDFLRSSYGFKHDFLGVVAAVIVAFAVVFAFTFALGIKAFNFQRR

SEQ ID NO: 3 - DNA, 40 bp, Artificial Sequence
  forward primer in example 3.1
aaaaagcaggctaccatgcccgaggccaagcttaacaata

SEQ ID NO: 4 - DNA, 40 bp, Artificial Sequence
  reverse primer of example 3.1
agaaagctgggtccatcttggtaagtttcttttcttaacc

SEQ ID NO: 5 - protein, 37 aa, Ipomoea batatas
  ref <301>: AUTHORS: Matsuoka and Nakamura
  ref <302>: TITLE: Propeptide of a precursor to a plant vacuolar protein required for vacuolar targeting
  ref <303>: JOURNAL: Proc. Natl. Acad. Sci. USA
  ref <304>: VOLUME: 88
  ref <305>: ISSUE: 3
  ref <306>: PAGES: 834-8
  ref <307>: DATE: 1991-02-01
  ref <308>: DATABASE ACCESSION NUMBER: PMID: 1992474
  ref <309>: DATABASE ENTRY DATE: 1991-02-01
  ref <313>: RELEVANT RESIDUES IN SEQ ID NO: (1)..(37)
MKAFTLALFLALSLYLLPNPAHSRFNPIRLPTTHEPA

SEQ ID NO: 6 - protein, 12 aa, Nicotiana tabacum
  ref <301>: AUTHORS: Neuhaus, J.M.; Sticher, L.; Meins, F. and Boller, T.
  ref <302>: TITLE: A short C-terminal sequence is necessary and sufficient for the targeting of chitinases to the plant vacuole
  ref <303>: JOURNAL: Proc. Natl. Acad. Sci. USA
  ref <304>: VOLUME: 88
  ref <305>: ISSUE: 22
  ref <306>: PAGES: 10362-10366
  ref <307>: DATE: 1991-11-15
  ref <308>: DATABASE ACCESSION NUMBER: PMID: 1946457
  ref <309>: DATABASE ENTRY DATE: 1991-11-25
  ref <313>: RELEVANT RESIDUES IN SEQ ID NO: (318)..(329)
DLLGNGLLVDTM

SEQ ID NO: 7 - DNA, 34 bp, Artificial Sequence
  forward primer in example 5.1
aaaaagcaggctaccatggagacgttatcgagaa

SEQ ID NO: 8 - DNA, 34 bp, Artificial Sequence
  reverse primer in example 5.1
agaaagctgggtctatcgttgttggaagttgagc

SEQ ID NO: 9 - DNA, 12789 bp, Artificial Sequence
  vector pK7WG2D
  misc_feature (4772)..(4772): n can be any base
tgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattatcagcttgcatgccggtcgatctagtaacatagatgacaccgcgcgcgataatttatcctagtttgcgcgctatattttgttttctatcgcgtattaaatgtataattgcgggactctaatcaaaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttgactctagctagagtccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccctgggtcacgacgagatcctcgccgtcgggcatccgcgccttgagcctggcgaacagttcggctggcgcgagcccctgatgctcttcgtccagatcatcctgatcgacaagaccggcttccatccgagtacgtcctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtatgataattcgcggtacccggggatcctctagagggcccgacgtcgcatgcctgcaggtcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcggactctagcatggccgcgggatatcaccactttgtacaagaaagctgaacgagaaacgtaaaatgatataaatatcaatatattaaattagattttgcataaaaaacagactacataatactgtaaaacacaacatatccagtcactatggtcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtatacatatcagtatatattcttataccgcaaaaatcagcgcgcaaatacgcatactgttatctggcttttagtaagccggatccacgcgtttacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgccggatcctaactcaaaatccacacattatacgagccggaagcataaagtgtaaagcctggggtgcctaatgcggccgccatagtgactggatatgttgtgttttacagtattatgtagtctgttttttatgcaaaatctaatttaatatattgatatttatatcattttacgtttctcgttcagcttttttgtacaaacttgtgatatcactagtgcggccgcctgcaggtcgactagaatagtaaattgtaatgttgtttgttgtttgttttgttgtggtaattgttgtaaaaatacggatcgtcctgcagtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgacgaagattttcttcttgtcattgagtcgtaaaagactctgtatgaactgttcgccagtcttcacggcgagttctgttagatcctcgatctgaatttttgactccatggcctttgattcagtaggaactactttcttagagactccaatctctattacttgccttggtttatgaagcaagccttgaatcgtccatactggaatagtacttctgatcttgagaaatatatctttctctgtgttcttgatgcagttagtcctgaatcttttgactgcatctttaaccttcttgggaaggtatttgatctcctggagattattactcgggtagatcgtcttgatgagacctgccgcgtaggcctctctaaccatctgtgggtcagcattctttctgaaattgaagaggctaatcttctcattatcggtggtgaacatggtatcgtcaccttctccgtcgaactttcttcctagatcgtagagatagagaaagtcgtccatggtgatctccggggcaaagganatctcgaccatatgggagagctcaagcttgcatgcctgcaggtcactggattttggttttaggaattagaaattttattgatagaagtattttacaaatacaaatacatactaagggtttcttatatgctcaacacatgagcgaaaccctataagaaccctaattcccttatgtgggaactactcacacattattctggagaaaaatagagagagatagatttgtagagagagactggtgatttttgcggactctagaactagtggatcccccgggctgcagccgggcggcgcttacagctcgtccttcttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatgtcggccgaggataatgataggagaagtgaaaagatgaaaaagagaaaaagattagtcttcaccatggctatcgttcgtaaatggtgaaaattttcagaaaatagcttttgctttaaaagaaatgatttaaattgctgcaatagaagtagaatgcttgattgcttgagattcgtttgttttgtatatgttgtgttgagaattcgagctcggtacccggggatcctctagcgaattttctctgctcaaattgttgaggttagcggatttgtaaacgcgtttatatgggctgcttggagggtacttttggattaatttttttctgccagcgcattctgacgcggcaccgctttggaaagtgcgctgtgggtccgcgttttctacaataatgtgccgatccggtcagaaagtatatggatgagttgtgccagcctcaccaacgtgctgcaggcccatcatgactacttcaatgttaatgggggtaatgaataaataggcgaaattgggttcacggtgggcccagggaatataatattgccgcagaggtagtcggatgccaaggcccgcaactaatagttcacgaacaaattcctagagagtcgacctgcagcatgcaagctaacctgcaggcatgcaagcttagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaat

SEQ ID NO: 10 - DNA, 20 bp, Artificial Sequence
  degenerate primer ALGG39
  misc_feature (3)..(3): n = Inosine
  misc_feature (9)..(9): n = Inosine
  misc_feature (11)..(11): n = Inosine
  misc_feature (15)..(15): g or a
  misc_feature (18)..(18): any base
ccnrgykcngnaaaracnac

SEQ ID NO: 11 - DNA, 20 bp, Artificial Sequence
  degenerate primer ALGG40
  misc_feature (3)..(3): n = Inosine
  misc_feature (15)..(15): any base
  misc_feature (18)..(18): any base
acnckyttyttytgnccncc

SEQ ID NO: 12 - DNA, 8 bp, Artificial Sequence
  degenerate primer ALGG41
  misc_feature (6)..(6): any base
  misc_feature (5)..(5): g or a
  misc_feature (3)..(3): any base
tcnarncc

SEQ ID NO: 13 - DNA, 23 bp, Artificial Sequence
  degenerate primer ALGG42
  misc_feature (3)..(3): n = Inosine
  misc_feature (6)..(6): n = Inosine
  misc_feature (9)..(9): n = Inosine
  misc_feature (12)..(12): n = Inosine
  misc_feature (15)..(15): any base
  misc_feature (18)..(18): any base
ggngtnytnacngcnytnatggg

SEQ ID NO: 14 - DNA, 23 bp, Artificial Sequence
  degenerate primer ALGG43
  misc_feature (3)..(3): any base
  misc_feature (18)..(18): any base
  misc_feature (5)..(5): g or a
  misc_feature (21)..(21): g or a
  misc_feature (12)..(12): n = Inosine
  misc_feature (15)..(15): n = Inosine
tcnarcatccangtngcnggrtt

SEQ ID NO: 15 - DNA, 8 bp, Artificial Sequence
  degenerate primer ALGG44
  misc_feature (6)..(6): g or a
ckccarta

SEQ ID NO: 16 - DNA, 4251 bp, Arabidopsis thaliana
atggagacgttatcgagaagcctaagtaagagcttaggagagttgttagcgagtaatagtaataaccatttctcaaggagaagcggttcaacgatagatgatcacgatgaggaagctcttaaatgggctgctctcgagaaacttccaacctttgctcgtcttcgtaccactatcattcatcctcacgaagatctcgtcgacgttaccaagctcggtgttgatgatcgccagaagttcatcgattctatcttcaaagtcaccgaggaagataacgagaagttcttgaaaaagtttaggaatcgaatcgacagggtgcggataaagctaccgacagtggaagtgagattcgagaaagtaacgatagaggcgaattgtcacataggaaaaagagctcttcccacattacccaacgctgctttaaacattgcagagagaggtcttcgtttgcttggttttaactttacaaaaaccactaaagtcactattcttagagacgtctccgggattatcaaaccttcaagaatgacacttttattaggtccaccatcatcagggaaaacaactcttttattagcgttagctggaaaactagaccaaagcttaaaggtgacagggagagtaacatacaatgggcatggattggaagagtttgtgccacagaaaacatcagcatacataagccaaaacgatgttcatgttggagtcatgactgtccaagaaactcttgatttttctgctagatgtcaaggagttggcactagatatgatttgttaagtgagttggtgagaagagagaaggacgcagggatcttgccggaaccggaagtggatctcttcatgaagtccattgcagctggaaatgtcaagagcagtctcatcacagactatactctcagaatattagggcttgacatatgcaaagatacggtggtcggagatgagatgattcgaggaatctcgggaggtcaaaagaaaagagtcacaacaggagagatgatagtaggaccaacaaagacattgttcatggatgagatatcgacaggtctagacagttcaacaacgtaccaaatagtgaaatgccttcaagagattgttcggttcacggacgcaacggttctcatgtccttattacaaccagctcctgaaacgttcgaacttttcgatgacatcattctattgtctgaaggacagatcgtgtaccaaggtccacgtgatcatgttcttaccttctttgaaacttgcggtttcaagtgtcccgaccgcaaaggcaccgcagatttcttgcaagaggtaacttcaaggaaagaccaagaacagtattgggcagactcaaaaaaaccatacagttacatttctgtctctgaattctccaaacgattcagaaccttccatgtcggagccaatcttgaaaaagatctatcagtcccttacgacagattcaaatcacacccagcttctctcgtcttcaagaaacactctgttccgaaatctcaactcttcaaggtctgttgggacagagaattgcttctcatgaaacgtaacgctttcttctacattaccaaaacagttcaaattatcattatggctttgattgcgtcgaccgtgtacctgagaacggagatgggaacaaagaacgagagtgatggagccgtctacatcggtgcgttgatgttctccatgatcgttaacatgtttaatggatttgcagagcttgctttgatgattcagagacttcccgtgttctacaagcaacgagaccttttgtttcatcctccttggaccttcagtcttccaacatttctcttgggtattcctatctccatcttcgaatctgttgtttgggtcaccattacttattacatgatcggatttgctccagaactcagcaggttcttgaagcatttgttggtgatatttctgacacagcaaatggctggtggtatatttaggttcatcgcggcaacttgcagatcgatgattcttgctaatacgggaggagctctagttatcctcctcctcttcttgcttggagggttcattgttcctagaggtgagatacctaaatggtggaaatgggcttactgggtttcaccaatggcttacacttacgatgctttaactgtcaatgaaatgcttgctcctagatggattaatcaaccgtcttctgataactccacgagcttagggttggctgttcttgagattttcgatattttcacggatccaaactggtattggatcggggtaggaggcattctcgggtttacggttctctttaacatcctcgttaccctcgctcttacattcctaaacccacttgagaaacagcaagccgttgtttcaaaagaaaatacagaggaaaacagagcagagaacggctcaaagagtaagagcattgacgtgaaaagaggaatggttctaccttttactcctctcacaatgtccttcgacaacgtaaactactacgtcgacatgcctaaggaaatgaaggaacaaggagtttccaaggataagcttcagctactaaaggaagtgacaggggtctttaggccaggggtattaacagctttgatgggagttagtggagctggtaagaccactttaatggatgttttagctggtagaaaaaccggtggttacatcgaaggagacatcagaatctctggtttccctaaaagacaagaaacctttgcaagaatctctggttattgtgaacagaacgatatccactcaccgcaagttactgtcaaagagtctctcatctactctgctttccttcgtctcccaaaagaagtcactaaatatgagaaaatgagatttgtggatgaagtgatggagctagtagagctagagagtctaaaagacgcggtggtggggcttccaggtatcacagggttatcaacagagcagaggaagagacttacaattgcagtggaattagtggcgaatccatctataatcttcatggatgagccaacctctggtcttgacgctagggctgcagcgattgtgatgaggacagtgaggaatacagttgacacaggaagaactgtggtctgcacaatccaccaacctagcattgatatctttgaagcttttgatgaacttcttcttttgaagagaggaggacaagtgatttatgctggtcctttaggtcaaaactctcacaagattatcgaatatttccaggcgattcatggagttccgaagattaaagagaagtataatccggcgacatggatgctagaagtgagctcaatggctgcagaagctaagcttgagatagattttgctgagcattataaaacatcatccttatatcaacaaaacaagaatcttgtaaaggaactaagcacaccaccacaaggagcaagcgatctctacttctcaacgcggttctcacaatcattgttaggacaattcaaatcttgtttgtggaagcagtggataacttactggagaactcctgactacaatctagcaagatttttctttacattggctgcagctgtcatgctcggatcaattttctggaaagttggcacaaaaagggaaaacgctaatgatcttacaaaggttattggagcaatgtatgctgctgttttattcgtgggcgtaaacaactcttcatctgtccaaccgctaatagccgtggaaagatctgtattctatagagaaagagctgctgaaatgtactctgctttaccttatgccctagcacaggtggtatgcgaaataccgtatgtgctgatccaaactacatattatacactaattatatacgccatgatgtgtttcgaatggacattggccaaattcttttggttttacttcgtatcatttatgtccttcctctacttcacatactatgggatgatgacagttgccctcaccccaaatcagcaagttgcagctgtttttgccggagctttctacggacttttcaacctcttttctggttttgtcattcctagaccgagaattccgaaatggtggatatggtattactggatctgtccagtggcttggactgtgtatggattgattgtttcgcagtacggtgacgtggaggatacgattaaagttccgggtatggcaaatgatccgacgataaagtggtacattgaaaaccactatggatacgacgcagattttatgataccgattgctactgtgttagtgggctttactctcttttttgcgtttatgtttgcttttggcataagaacgctcaacttccaacaacgatag

SEQ ID NO: 17 - protein, 1416 aa, Arabidopsis thaliana
METLSRSLSKSLGELLASNSNNHFSRRSGSTIDDHDEEALKWAALEKLPTFARLRTTIIHPHEDLVDVTKLGVDDRQKFIDSIFKVTEEDNEKFLKKFRNRIDRVRIKLPTVEVRFEKVTIEANCHIGKRALPTLPNAALNIAERGLRLLGFNFTKTTKVTILRDVSGIIKPSRMTLLLGPPSSGKTTLLLALAGKLDQSLKVTGRVTYNGHGLEEFVPQKTSAYISQNDVHVGVMTVQETLDFSARCQGVGTRYDLLSELVRREKDAGILPEPEVDLFMKSIAAGNVKSSLITDYTLRILGLDICKDTVVGDEMIRGISGGQKKRVTTGEMIVGPTKTLFMDEISTGLDSSTTYQIVKCLQEIVRFTDATVLMSLLQPAPETFELFDDIILLSEGQIVYQGPRDHVLTFFETCGFKCPDRKGTADFLQEVTSRKDQEQYWADSKKPYSYISVSEFSKRFRTFHVGANLEKDLSVPYDRFKSHPASLVFKKHSVPKSQLFKVCWDRELLLMKRNAFFYITKTVQIIIMALIASTVYLRTEMGTKNESDGAVYIGALMFSMIVNMFNGFAELALMIQRLPVFYKQRDLLFHPPWTFSLPTFLLGIPISIFESVVWVTITYYMIGFAPELSRFLKHLLVIFLTQQMAGGIFRFIAATCRSMILANTGGALVILLLFLLGGFIVPRGEIPKWWKWAYWVSPMAYTYDALTVNEMLAPRWINQPSSDNSTSLGLAVLEIFDIFTDPNWYWIGVGGILGFTVLFNILVTLALTFLNPLEKQQAVVSKENTEENRAENGSKSKSIDVKRGMVLPFTPLTMSFDNVNYYVDMPKEMKEQGVSKDKLQLLKEVTGVFRPGVLTALMGVSGAGKTTLMDVLAGRKTGGYIEGDIRISGFPKRQETFARISGYCEQNDIHSPQVTVKESLIYSAFLRLPKEVTKYEKMRFVDEVMELVELESLKDAVVGLPGITGLSTEQRKRLTIAVELVANPSIIFMDEPTSGLDARAAAIVMRTVRNTVDTGRTVVCTIHQPSIDIFEAFDELLLLKRGGQVIYAGPLGQNSHKIIEYFQAIHGVPKIKEKYNPATWMLEVSSMAAEAKLEIDFAEHYKTSSLYQQNKNLVKELSTPPQGASDLYFSTRFSQSLLGQFKSCLWKQWITYWRTPDYNLARFFFTLAAAVMLGSIFWKVGTKRENANDLTKVIGAMYAAVLFVGVNNSSSVQPLIAVERSVFYRERAAEMYSALPYALAQVVCEIPYVLIQTTYYTLIIYAMMCFEWTLAKFFWFYFVSFMSFLYFTYYGMMTVALTPNQQVAAVFAGAFYGLFNLFSGFVIPRPRIPKWWIWYYWICPVAWTVYGLIVSQYGDVEDTIKVPGMANDPTIKWYIENHYGYDADFMIPIATVLVGFTLFFAFMFAFGIRTLNFQQR

(19) United States
(12) Patent Application Publication
(10) Pub. No.: US 2010/0170010 A1
(43) Pub. Date: Jul. 1, 2010

(54) Use of genes encoding membrane transporter pumps to stimulate the production of secondary metabolites in biological cells

(76) Inventors: Alain Goossens, Lokeren (BE); Dirk G Inzé, Moorsel-Aalst (BE)

Correspondence Address: TRASKBRITT, P.C., P.O. BOX 2550, SALT LAKE CITY, UT, 84110, US

(21) Appl. No.: 12/592,505

(22) Filed: Nov. 24, 2009

Related U.S. Application Data
(63) Continuation-in-part of application No. 10/666,778, filed on Sep. 18, 2003, now Pat. No. 7,622,634.
Application No. 10/666,778 is a continuation of application No. PCT/EP02/04322, filed on Apr. 18, 2002.

(30) Foreign Application Priority Data
Apr. 18, 2001 (EP) 01201407.2

(51) Int. Cl. A01H 5/00; C12P 13/00; C12N 15/82; C12N 5/10; C07H 21/04

(52) U.S. Cl. 800/298; 435/128; 435/468; 435/419; 536/23.6

(57) ABSTRACT

The invention relates to the field of secondary metabolite production in plants and plant cell cultures. More specifically, the invention relates to the use of transporters and more particularly ABC-transporters to enhance the production and/or secretion of secondary metabolites in plants and plant cell cultures.

### CROSS-REFERENCE TO RELATED APPLICATIONS

This application is a continuation-in-part of co-pending U.S. application Ser. No. 10/666,778, filed Sep. 18, 2003, which itself is continuation of PCT International Patent Application No. PCT/EP/02/04322, filed on Apr. 18, 2002, designating the United States of America, and published, in English, as PCT International Publication No. WO 02/083888 A2 on Oct. 24, 2002, which claims priority under Article 8 of the PCT to EP 0/201407.2. This application is also a continuation-in-part of PCT International Patent Application No. PCT/EP/02/04322. The contents of the entirety of each of which is incorporated by this reference.

### TECHNICAL FIELD

The invention relates generally to biotechnology, and more specifically to the field of secondary metabolite production in plants and plant cell cultures. Particularly, the invention relates to the use of transporters and more particularly ABC-transporters to enhance the production and/or secretion of secondary metabolites in plants and plant cell cultures.

### BACKGROUND

Higher plants are able to produce a large number of small-molecular-weight compounds with very complex structures. These compounds, called “secondary metabolites”, can play a role in the resistance against pests and diseases, attraction of pollinators and interaction with symbiotic microorganisms. Besides the importance for the plant itself, secondary metabolites are of great commercial interest because they determine the quality of food (color, taste, and aroma) and ornamental plants (flower color, smell). A number of secondary metabolites isolated from plants are commercially available as fine chemicals, for example, drugs, dyes, flavours, fragrances and even pesticides. In addition, various health improving effects and disease preventing activities of secondary metabolites have been discovered, such as anti-oxidative and anti-metastatic-lowering properties (e.g., vinblastine, taxol).

Although about 100,000 plant secondary metabolites are already known, only a small percentage of all plants have been studied to the extent necessary for the determination of the presence of secondary metabolites. It is expected that interest in such metabolites will continue to grow as for example, plant sources of new and useful drugs are discovered. Some of these valuable phytochemicals are quite expensive because they are only produced at extremely low levels in plants.

Very little is known about the biosynthesis of secondary metabolites in plants. However, some recently elucidated biosynthetic pathways of secondary metabolites are long and complicated requiring multiple enzymatic steps to produce the desired end product. Most often, the alternative of producing these secondary metabolites through chemical synthesis is complicated due to a large number of asymmetric carbons and in most cases chemical synthesis is not economically feasible.

The recovery of valuable secondary metabolites is mostly achieved through extraction and purification (generally at low yields) of imported, sometimes exotic, plant biomasses, whose reproductive agriculture and secure long term supply are often very difficult, if not impossible to guarantee. The problems of obtaining useful metabolites from natural sources may potentially be circumvented by cell culture. The culture of plant cells has been explored since the 1960's as a viable alternative for the production of complex phytochemicals of industrial interest. Although plant cell cultures might be somewhat sensitive for shear forces, many cultures can be grown in large bioreactors without difficulty. For example, the use of large-scale plant cell cultures in bioreactors for the production of alkaloids has been extensively studied (Verpoorte et al. (1999) Biotechnol. Lett. 21, 467). Since it has been observed that undifferentiated cultures such as callus and cell suspension cultures produce only very low levels of secondary metabolites one tends to use differentiated plant cell cultures such as root- and hairy root-culture. For example, tropane alkaloids that are only scarcely synthesized in undifferentiated cells are produced at relatively high levels in cultured roots.

Despite the promising features and developments, the production of plant-derived pharmaceuticals by plant cell cultures has not been fully commercially exploited. The main reasons for this reluctance shown by industry to produce secondary metabolites by means of cell cultures, compared to the conventional extraction of whole plant material, are economical ones based on the slow growth and the low production levels of secondary metabolites by such plant cell cultures. Important causes are the toxicity of such compounds to the plant cell, and the role of catabolism of the secondary metabolites. Another important problem is that secondary metabolites are mostly retained intracellularly complicating the downstream processing and purification. Indeed, often laborious extraction schemes have to be developed for each specific secondary metabolite of interest.

### DISCLOSURE OF THE INVENTION

The invention provides a solution to these problems. The invention uses genes encoding ABC-transporters to enhance the production of secondary metabolites in plant cell cultures. In embodiments, the enhanced production or secretion of the secondary metabolite may be extracellular production or secretion. ABC-transporters are well-known in the field of cancer therapy as molecular ‘pumps’ in tumour-cell membranes that actively expel chemotherapy drugs from the interior of the cells. This allows tumour cells to avoid the toxic effects of the drug or molecular processes within the nucleus or the cytoplasm.

The two pumps commonly found to confer chemoresistance in cancer are P-glycoprotein and the so-called multidrug resistance-associated protein (MRP). In addition, ABC-transporters have been used in plants as a selection marker (PCT International Patent Publication WO 99/10514) and for the protection of plants for the detrimental effects of certain exogenously added xenobiotics (PCT International Patent Publication No. WO 00/18886, Muhitch J. M. et al. (2000) Plant Science, 157, 201). In U.S. Pat. No. 6,166,290, it is shown that the use of ABC-transporters in plants can be used to stimulate remediation, to strengthen the disease response and to modulate plant pigmentation. It has, however, never been shown in the art that ABC-transporters can be used to enhance the level of secondary metabolites made in plant cell cultures neither has it been shown that ABC-transporters can be used to stimulate the secretion of endogenously synthesized secondary metabolites from the inside of plant cells to the extracellular space.

### BRIEF DESCRIPTION OF THE FIGURES

FIG. 1: Plasmid map of the pK7WGD2 binary vector.

FIG. 2: Hyoscyamine-induced cell death in transformed BY-2 cells. Three-day old transformed BY-2 cell cultures were incubated in the absence (CON) or presence (HYO) of 30 mM hyoscyamine for 24 hours. Cell death was assayed at two time points (6 hours and 24 hours) by Evans blue staining and is indicated as the fold increase in optical density at OD₆₀₀ relative to the value at the start of the experiment. Values are the mean of three independent experiments. GUS, US50, W303 and AT represent BY-2 cell lines transformed with pK7WGD2-GUS, pK7WGD2-ScPDR5-US50, pK7WGD2-ScPDR5-W303 and pK7WGD2-AtPDR1 respectively.

FIG. 3: HmPDR1 expression is induced by CdCl₂. Quantitative RT-PCR analysis of HmPDR1 in total RNA from H. muticus hairy roots treated with 1 mM CdCl₂ or H₂O as a control. Ethidium bromide-stained rRNA is used as a control. The fold increase in the ratio of HmPDR1 transcript to rRNA fluorescence, relative to the value at time point zero, is given below the panels. Time after elicitation is indicated in hours.

### DETAILED DESCRIPTION OF THE INVENTION

The present invention accordingly provides in one embodiment a method for inducing or enhancing the production or the secretion of at least one secondary metabolite in biological cells by transformation of the biological cells with an expression vector comprising an expression cassette that further comprises a gene coding for a transporter. In embodiments, the enhanced production or secretion of the secondary metabolite may be extracellular production or secretion. With “at least one secondary metabolite” it is meant related structures of secondary metabolites and intermediates or precursors thereof. The biological cells can be plant cells, fungal cells, bacteria cells, algae cells and/or animal cells. A “transporter” is a protein capable of interacting with at least one specific secondary metabolite and transporting the metabolite across a membrane wherein the membrane comprises the vacuolar membrane (tonoplast), or chloroplast membrane or plasma membrane. The transporter gene can be heterologous or homologous to the biological cell.

“Expression cassettes”, of the present invention are generally DNA constructs preferably including (5′ to 3′ in the direction of transcription): a promoter region, a gene encoding for a transporter operatively linked with the transcription initiation region, and a termination sequence including a stop signal for RNA polymerase and a polyadenylation signal. It is understood that all of these regions should be capable of operating in the biological cells to be transformed. The promoter region comprising the transcription initiation region, which preferably includes the RNA polymerase binding site, and the polyadenylation signal may be native to the biological cell to be transformed or may be derived from an alternative source, where the region is functional in the biological cell.

The transporters of this invention may be expressed in for example a plant cell under the control of a promoter that directs constitutive expression or regulated expression. Regulated expression comprises temporally or spatially regulated expression and any other form of inducible or repressible expression. “Temporally” means that the expression is induced at a certain time point, for instance, when a certain growth rate of the plant cell culture is obtained (e.g., the promoter is induced only in the stationary phase or at a certain stage of development).

“Spatially” means that the promoter is only active in specific organs, tissues, or cells (e.g., only in roots, leaves, epidermis, guard cells or the like. Other examples of regulated expression comprise promoters whose activity is induced or repressed by adding chemical or physical stimuli to the plant cell. In a preferred embodiment, the expression of the transporters is under control of environmental, hormonal, chemical, and/or developmental signals, also can be used for expression of transporters in plant cells, including promoters regulated by (1) heat, (2) light, (3) hormones, such as abscisic acid and methyl jasmonate (4) wounding or (5) chemicals such as salicylic acid, chitosans or metals. Indeed, it is well known that the expression of secondary metabolites can be boosted by the addition of for example specific chemicals, jasmonate and elicitors. The co-expression of transporters, in combination with a stimulation of secondary metabolite synthesis is beneficial for an optimal and enhanced production or secretion of secondary metabolites. Alternatively, the transporters can be placed under the control of a constitutive promoter. A constitutive promoter directs expression in a wide range of cells under a wide range of conditions. Examples of constitutive plant promoters useful for expressing heterologous polypeptides in plant cells include, but are not limited to, the cauliflower mosaic virus (CaMV) 35S promoter, which confers constitutive, high-level expression in most plant tissues including monocots; the nopaline synthase promoter and the octopine synthase promoter.

The expression cassette is usually provided in a DNA or RNA construct which is typically called an “expression vector” which is any genetic element, for example, a plasmid, a chromosome, a virus, behaving either as an autonomous unit of polynucleotide replication within a cell (i.e., capable of replication under its own control) or being rendered capable of replication by insertion into a host cell chromosome, having attached to it another polynucleotide segment, so as to bring about the replication and/or expression of the attached segment. Suitable vectors include, but are not limited to, plasmids, bacteriophages, cosmids, plant viruses and artificial chromosomes. The expression cassette may be provided in a DNA construct which also has at least one replication system. In addition to the replication system, there will frequently be at least one marker present, which may be useful in one or more hosts or different markers for individual hosts. The markers may a) code for protection against a biocide, such as antibiotics, toxins, heavy metals, certain sugars or the like; b) provide complementation, by imparting prototrophy to an auxotrophic host: or c) provide a visible phenotype through the production of a novel compound in the plant.

Exemplary genes which may be employed include neomycin phosphotransferase (NPTII), hygromycin phosphotransferase (HPT), chloramphenicol acetyltransferase (CAT), nitrilase, and the gentamycin resistance gene. For plant host selection, non-limiting examples of suitable markers are β-glucuronidase, providing indigo production, luciferase, providing visible light production, Green Fluorescent Protein and variants thereof, NPTII, providing kanamycin resistance or G418 resistance, HPT, providing hygromycin resistance, and the mutated aroA gene, providing glyphosate resistance.

The term “promoter activity” refers to the extent of transcription of a gene that is operably linked to the promoter whose promoter activity is being measured. The promoter activity may be measured directly by measuring the amount of RNA transcript produced, for example by Northern blot or indirectly by measuring the product coded for by the RNA transcript, such as when a reporter gene is linked to the promoter.

The term “operably linked” refers to linkage of a DNA segment to another DNA segment in such a way as to allow the segments to function in their intended manners. A DNA sequence encoding a gene product is operably linked to a regulatory sequence when it is ligated to the regulatory sequence, such as, for example a promoter, in a manner which allows modulation of transcription of the DNA sequence, directly or indirectly. For example, a DNA sequence is operably linked to a promoter when it is ligated to the promoter downstream with respect to the transcription initiation site of the promoter and allows transcription elongation to proceed through the DNA sequence. A DNA for a signal sequence is operably linked to DNA coding for a polypeptide if it is expressed as a pre-protein that participates in the transport of the polypeptide. Linkage of DNA sequences to regulatory sequences is typically accomplished by ligation at suitable restriction sites or adapters or linkers inserted in lieu thereof using restriction endonucleases known to one of skill in the art.

The term “heterologous DNA” or “heterologous RNA” refers to DNA or RNA that does not occur naturally as part of the genome or DNA or RNA sequence in which it is present, or that is found in a cell or location in the genome or DNA or RNA sequence that differs from that which is found in nature. Heterologous DNA and RNA (in contrast to homologous DNA and RNA) are not endogenous to the cell into which it is introduced, but has been obtained from another cell or synthetically or recombinantly produced. An example is a human gene, encoding a human protein, operably linked to a non-human promoter. Another example is a gene isolated from one plant species operably linked to a promoter isolated from another plant species. Generally, though not necessarily, such DNA encodes RNA and proteins that are not normally produced by the cell in which the DNA is transcribed or expressed. Similarly exogenous RNA encodes for proteins not normally expressed in the cell in which the exogenous RNA is present. Heterologous DNA or RNA may also refer to as foreign DNA or RNA. Any DNA or RNA that one of skill in the art would recognize as heterologous or foreign to the cell in which it is expressed is herein encompassed by the term heterologous DNA or heterologous RNA. Examples of heterologous DNA include, but are not limited to, DNA that encodes proteins, polypeptides, receptors, reporter genes, transcriptional and translational regulatory sequences, selectable or traceable marker proteins, such as a protein that confers drug resistance, RNA including mRNA and antisense RNA and ribozymes.

Generally, two basic types of metabolites are synthesized in cells, i.e. those referred to as primary metabolites and those referred to as secondary metabolites. A primary metabolite is any intermediate in, or product of the primary metabolism in cells. The primary metabolism in cells is the sum of metabolic activities that are common to most, if not all, living cells and are necessary for basal growth and maintenance of the cells. Primary metabolism thus includes pathways for generally modifying and synthesizing certain carbohydrates, proteins, fats and nucleic acids, with the compounds involved in the pathways being designated primary metabolites. In contrast hereto, secondary metabolites usually do not appear to participate directly in growth and development. They are a group of chemically very diverse products that often have a restricted taxonomic distribution. Secondary metabolites normally exist as members of closely related chemical families, usually of a molecular weight of less than 1500 Dalton, although some bacterial toxins are considerably longer. Secondary plant metabolites include, for example, alkaloid compounds (e.g., terpenoid indole alkaloids, tropane alkaloids, steroid alkaloids, polyhydroxy alkaloids), phenolic compounds (e.g., quinines, lignans and flavonoids), terpenoid compounds (e.g., monoterpenoids, iridoids, sesquiterpenoids, diterpenoids and triterpenoids). In addition, secondary metabolites include small molecules (i.e., those having a molecular weight of less than 600), such as substituted heterocyclic compounds which may be monocyclic or polycyclic, fused or bridged. Many plant secondary metabolites have value as pharmaceuticals. Plant pharmaceuticals include, for example, taxol, digoxin, colchicines, codeine, morphine, quinine, shikonin, ajmalicine, and vinblastine.

The definition of “alkaloids”, of which more than 12,000 structures have been described already, includes all nitrogen-containing natural products which are not otherwise classified as peptides, non-protein amino acids, amines, cyanogenic glycosides, glucosinolates, cofactors, phytohormones or primary metabolites (such as purine and pyrimidine bases). The “calystegins” constitute a unique subgroup of the tropane alkaloid class (Goldmann et al. (1990) Phytochemistry, 29, 2125). They are characterized by the absence of an N-methyl substituent and a high degree of hydroxylation. TrIhydroxylated calystegins are summarized as the calystegin A-group, tetrahydroxylated calystegins as the B-group, and pentahydroxylated derivates form the C-group. Calystegins represent a novel structural class of polyhydroxy alkaloids possessing potent glycosidase inhibitory properties next to longer known classes of the monocyclic pyrrolidones (e.g., dihydroxymethyldihydroxy pyrrolidine) pyrrolines and piperidines (e.g., deoxynojirimycin), and the bicyclic pyrrolizidines (e.g., australine) and indolizidines (e.g., swainsonine and castanospermine). Glycosidase inhibitors are potentially useful as antidiabetic, antiviral, antimetastatic, and immunomodulatory agents.

In another embodiment, the invention provides a method for enhancing the production of at least one secondary metabolite in biological cells by transformation of the biological cells with an expression vector comprising an expression cassette further comprising a gene coding for an ABC transporter. In embodiments the enhanced production of the secondary metabolite may be enhanced levels of the secondary metabolite that are extracellular. Genes useful to be incorporated in an expression cassette for carrying out the present invention include those coding for ATP-binding cassette (ABC) transporters. Genes encoding ABC-transporters can be of any species or origin, including microorganisms, plant and animal (Higgins (1992) Ann. Rev. Cell Biol. 8, 67), but are preferably of plant or fungal origin. The ATP-binding cassette (ABC) transporters, also called the “traffic ATPases”, comprise a superfamily of membrane proteins that mediate transport and channel functions in prokaryotes and eukaryotes (Higgins, C. F. (1992) Annu. Rev. Cell Biol. 8:67-113; Theodoulou F. (2000) Biochimica et Biophysica Acta 1465, 79). Typically, an ABC transporter contains two copies each of two structural units: a highly hydrophobic transmembrane domain (TMD), and a peripherally located ATP binding domain or nucleotide binding fold (NBF), which together are often necessary and sufficient to mediate transport. The TMD domains form the pathway via which the substrate crosses the membrane, and in some cases, have been shown to contribute to the substrate specificity. The NBFs are oriented towards the cytoplasmic side of the membrane and couple ATP hydrolysis to transport. Within the NBF is a conserved region of approximately 200 amino acids, consisting of the Walker A and B boxes separated by the ABC signature motif. It is this signature motif which distinguishes ABC transporters from other NTP binding proteins, such as the kinases, which also contain the Walker sequences. Sequence homology over the whole gene can be negligible between different ABC transporters, but in the conserved areas of the NBF it is typically 30-40% between family members, and this has proved useful in the isolation of ABC genes by approaches such as PCR and hybridization with degenerate nucleotides (Dudler R. et al (1998) Methods Enzymol. 292, 162). A great variety of specific substrates is transported by members of this family of transport proteins, including drugs, anorganic ions, amino acids, proteins, sugars, and polysaccharides. Eukaryotic ABC proteins include: P-glycoproteins, also known as multidrug resistance (MDR) proteins, which are associated with resistance to a wide range of hydrophobic drugs (MDR1; Gottesman, M. M. & Pastan, I. (1993) Annu. Rev. Biochem. 62:385-427) or with phosphatidyl choline transport (MDR2; Ruetz, S. & Gros, P. (1994) Cell 77:1071-1081); CFTR, the cystic fibrosis transmembrane conductance regulator (Welsh, M. J. & Smith, A. E. (1993) Cell 73:1251-1254); TAP proteins, the transporters associated with antigen processing in mammalian cells (Androlewicz, M. J. et al. (1994) Proc. Natl. Acad. Sci. USA 91:12716-12720); cMOAT/cMRP1, which is associated with transport of glutathione, glucuronide, and sulfate conjugates across the canalicular membrane (Buchler, M. et al. (1996) J. Biol. Chem. 271:15091-15098); and STE6, which exports the a-factor mating pheromone of S. cerevisiae (Michaelis, S. (1993) Semin. Cell Biol. 4:17-27) and PDR5, the pleiotropic drug resistance protein of yeast. Prokaryotic ABC proteins include periplasmic nutrient permeases, such as those responsible for uptake of maltose (MalFGK) and histidine (HisMPQ) in gram-negative bacteria, and toxin exporters such as those required for export of hemolysin (HlyB) and colicin (ColV) from E. coli. Sequence comparisons between MRP1 and other ABC transporters reveal two major subgroups among these proteins (Szczypka et al. (1994) J. Biol. Chem. 269, 22853). One subgroup comprises MRP1, the Saccharomyces cerevisiae cadmium factor (YCF1) gene, the Leishmania P-glycoprotein-related molecule (Lei/PgpA) and the CFTRs. The other subgroup comprises the multiple drug resistance proteins (MDRs), MHC transporters and STE6. Homologues of ABC-transporters have been identified in plant species. In Arabidopsis thaliana, the glutathione-conjugate transporter (MRP) is located in the vacuolar membrane and is responsible for sequestration of xenobiotics in the central vacuole. An MDR-like gene (atpgp1) has also been identified in A. thaliana, which encodes a putative P-glycoprotein homolog. This atpgp1 gene was found to share significant sequence homology and structural organization with human MDR genes. Other MDR homologues have been found in potato and barley. Genes encoding ABC-transporters of the present invention which may be operably linked with a promoter for expression in a plant species may be derived from a chromosomal gene, cDNA, a synthetic gene, or combinations thereof.

In another embodiment, DNA sequences encoding ABC-transporters are used to enhance the production of at least one secondary metabolite in plant cells comprising the transformation of the plant cells with an expression vector comprising an expression cassette further comprising a gene coding for an ABC-transporter.

By the term “enhanced production” it is meant that the level of one or more metabolites may be enhanced by at least 20%, 30%, 40%, 50%, 60%, 70%, 80%, 90% or at least 100% relative to the untransformed plant cell which was used to transform with an expression vector comprising an expression cassette further comprising a gene coding for a transporter or an ABC-transporter. In embodiments, enhanced production of a secondary metabolite may result in a detection of a higher level of secondary metabolites in the extracellular medium of the plant cell culture. In embodiments, a higher level of secondary metabolites may be detected inside the plant cells, for example in the vacuole.

The present invention can be practiced with any plant variety for which cells of the plant can be transformed with an expression cassette of the current invention and for which transformed cells can be cultured in vitro. Suspension culture, callus culture, hairy root culture, shoot culture or other conventional plant cell culture methods may be used (as described in: Drugs of Natural Origin, G. Samuelsson, 1999, ISBN 9186274813).

By “plant cells” it is understood any cell which is derived from a plant and can be subsequently propagated as callus, plant cells in suspension, organized tissue and organs (e.g., hairy roots).

Tissue cultures derived from the plant tissue of interest can be established. Methods for establishing and maintaining plant tissue cultures are well known in the art (see, e.g., Trigiano R. N. and Gray D. J. (1999), “Plant Tissue Culture Concepts and Laboratory Exercises”, ISBN: 0-8493-2029-1; Herman E. B. (2000), “Regeneration and Micropropagation: Techniques, Systems and Media 1997-1999”, Agricell Report). Typically, the plant material is surface-sterilized prior to introducing it to the culture medium. Any conventional sterilization technique, such as chlorinated bleach treatment can be used. In addition, antimicrobial agents may be included in the growth medium. Under appropriate conditions plant tissue cells form callus tissue, which may be grown either as solid tissue on solidified medium or as a cell suspension in a liquid medium.

A number of suitable culture media for callus induction and subsequent growth on aqueous or solidified media are known. Exemplary media include standard growth media, many of which are commercially available (e.g., Sigma Chemical Co., St. Louis, Mo.). Examples include Schenk-Hildebrandt (SH) medium, Linsmaier-Skoog (LS) medium, Murashige and Skoog (MS) medium, Gamborg's B5 medium, Nitsch & Nitsch medium, White's medium, and other variations and supplements well known to those of skill in the art (see, e.g., Plant Cell Culture, Dixon, ed. IRL Press, Ltd. Oxford (1985) and George et al., Plant Culture Media, Vol. 1, Formulations and Uses Exegetics Ltd. Wilts, UK, (1987)). For the growth of conifer cells, particularly suitable media include ½ MS, ½ L. P., DCR, Woody Plant Medium (WPM), Gamborg's B5 and its modifications, DV (Durzan and Ventimiglia, In Vitro Cell Dev. Biol. 30:219-227 (1994)), SH, and White's medium.

When secondary metabolites are produced in plant cell culture systems they usually have to be extracted and purified from the isolated plant cell mass which is an expensive process. It is known that plants can be made by means of genetic manipulation to store proteins in seed endosperm, from where they can be more easily extracted. It has also been described that some plant cells can secrete secondary metabolites can be secreted and that the secretion can be enhanced by for example the addition of elicitors (Kneer et al. (1999) J. Exp. Bot. 50, 1553) or by the addition of specific chemicals (Lee et al. (1998) Phytochemistry 49, 2342). It has however never been described that the secretion of secondary metabolites by plant cells can be induced or enhanced by the transformation of at least one specific gene into a plant cell. The present invention provides a solution for this problem by transformation of plant cells, producing secondary metabolites, with an expression cassette comprising a gene encoding an ABC-transporter. Therefore, in another embodiment of the invention, a DNA sequence encoding an ABC-transporter can be used to induce or enhance the secretion of at least one secondary metabolite produced in plant cell cultures comprising transforming the plant cells that are producing secondary metabolites, with an expression vector comprising an expression cassette further comprising a gene coding for an ABC-transporter, and selecting transformed plant cells with an induced or enhanced secretion of at least one secondary metabolite. Such transformed plant cells can be subsequently propagated using methods described herein before.

An “enhanced secretion of at least one secondary metabolite” means that there exists already a detectable secretion of the secondary metabolite(s) in the extracellular medium of the plant cell culture and that an increase of the secondary metabolite(s) can be measured by at least 20%, 30%, 40%, 50%, 60%, 70%, 80%, 90% or more than 90% compared to basal secretion by the untransformed plant cell culture. An “enhanced secretion” does not necessarily mean that there is a higher production, it can also mean that there is exists the same level of production but that the secretion is enhanced. In embodiments, cells with enhanced secretion may result in higher extracellular levels of the secondary metabolite when compared to cells without enhanced secretion.

An “induced secretion of at least one secondary metabolite” means that there is no detectable secretion of the secondary metabolite(s) in the extracellular medium of the untransformed plant cell culture but that the detection becomes possible upon carrying out the transformation according to the invention.

Generally, secondary metabolites can be measured, intracellularly or in the extracellular space, by methods known in the art. Such methods comprise analysis by thin-layer chromatography, high pressure liquid chromatography, capillary chromatography, (gas chromatographic) mass spectrometric detection, radioimmunoassay (RIA) and enzyme immuno-assay (ELISA).

In order to make clear what is meant by the word “secretion” in the current invention one has to make a clear distinction between the secretion of proteins which is mediated by an amino-terminal signal peptide and the secretion of secondary metabolites which is independent of an amino-terminal leader sequence. As the term is used herein, secretion means secretion of a secondary metabolite across the plasma membrane or secretion across both the plasma membrane and the cell wall of a plant cell. It should be noted that, in the scientific literature the term “secretion” often is used to indicate secretion into the apoplastic space, i.e., secretion across the plasma membrane but not across the cell wall.

In one aspect of the invention, there is no secretion of (a) secondary metabolite(s) into the growth medium. Then, the secretion can be induced by several possibilities: (1) by the transformation of the plant cell with a heterologous gene encoding an ABC-transporter or (2) by the overexpression of a homologous ABC-transporter which expressing is rate-limiting in the plant cell or (3) by the relocalization of a homologous or heterologous ABC-transporter from a vacuolar localization towards a membrane localization. In plants, proteins destined for the vacuole are sorted away from proteins destined for secretion at the trans-Golgi network, a process that requires the presence of positive sorting signals on the vacuolar proteins. Three types of sorting signals have been described for soluble vacuolar proteins in plants (Matsuoka and Neuhaus (1999) J. Exp. Botany 50, 165). Some proteins contain a cleavable amino-terminal propeptide that functions as a sorting signal while others contain a cleavable carboxy-terminal propeptide. Finally, a minor amount of plant proteins contains an internal vacuolar targeting determinant. According to the invention a homologous or heterologous ABC-transporter that is normally localized in the vacuolar membrane can be engineered by clipping off its vacuolar localization signal (carboxy-terminal or amino-terminal propeptide) or by deleting its internal vacuolar targeting determinant. If necessary a heterologous or homologous amino-terminal leader sequence is spliced to the gene encoding the homologous or heterologous ABC-transporter in order to provide entry into the secretion system. As a result the engineered ABC-transporter is not directed anymore in the secretion pathway towards its normal vacuolar localization but is deviated towards the extracellular space. However, due to the hydrophobic transmembrane signal present in ABC-transporters, the ABC-transporter is not secreted into the extracellular medium but remains sequestered into the plasma membrane of the plant cell. We show in the present invention that the novel intracellular localization of the ABC-transporter (from the vacuole to the plasma membrane) results in a secretion of the produced secondary metabolites into the medium of the plant cell culture.

In another aspect of the invention, there is already an existing, but low, level of secretion of (a) secondary metabolite(s) by the plant cell and then the secretion can be enhanced by (1) by the transformation of the plant cell with a heterologous gene encoding an ABC-transporter or (2) by the overexpression of a homologous ABC-transporter which expressing is rate-limiting in the plant cell or (3) by the relocalization of a homologous or heterologous ABC-transporter from a normal vacuolar localization towards a membrane localization.

In yet another aspect, an intermediary product of the secondary metabolite, which causes negative feedback inhibition on an enzymatic reaction step involved in the biosynthesis of the secondary metabolite, can be secreted by (1) by the transformation of the plant cell with a heterologous gene encoding an ABC-transporter or (2) by the overexpression of a homologous ABC-transporter which expressing is rate-limiting in the plant cell or (3) by the relocalization of a homologous or heterologous ABC-transporter from a vacuolar localization towards a membrane localization. The secretion of the intermediary product or an amount produced thereof reduces the negative feedback inhibition and consequently enhances the production of the secondary metabolite in the plant cell. The enhanced production of the secondary metabolite can be made secreted by the plant cell by the transformation of the already transformed plant cell with a second expression cassette comprising a gene encoding an ABC transporter, according to the method described above. In this case of secretion, the directed secondary metabolites can be easily isolated from the surrounding medium since they are directed into the extracellular space. Consequently, the breaking up of the cells that is necessary in the case of intracellular production can be omitted.

In another embodiment, the production of secondary metabolites can be enhanced by stimulating the transport of secondary metabolites into the vacuole. In plants, the targeting of proteins and compounds into the vacuole is of particular interest (especially from the point of view of application) because the vacuole is the largest storage compartment in the cell for reserve substances, detoxification products and defense substances. The most important storage takes place in vacuoles in plant organs such as tubers, bulbs, roots and stems. Similar considerations also apply to substances that can be used in the control of pests or diseases, especially when those substances prove to be toxic to the plant itself. Indeed, in certain cases the vacuole also serves as a detoxification organelle by, for example, storing the detoxification products synthesized by the plant. According to the present invention secondary metabolites can also be made secreted into the vacuole (1) by the transformation of a plant cell with a heterologous gene encoding an ABC-transporter or (2) by the overexpression of a homologous ABC-transporter which expressing is rate-limiting in the plant cell or (3) by the relocalization of a homologous or heterologous ABC-transporter from a normally localized plasma membrane localization towards a vacuolar localization. To perform the relocalization it is necessary to modify the gene encoding an ABC-transporter by genetically fusing it to an amino-terminal or carboxy-terminal vacuolar localization signal or by the genetic modification through the introduction of an existing internal vacuolar localization signal. U.S. Pat. No. 6,054,637 provides detailed information of genetic modification of genes through the addition or clipping off plant vacuolar localization signals. We observe that the secretion or targeting of the produced secondary metabolites into the vacuole reduces the toxicity to the plant cell.

In yet another embodiment, an intermediary product of the secondary metabolite, which causes negative feedback inhibition on an enzymatic reaction step involved in the biosynthesis of the secondary metabolite, can be made sequestered into the vacuole by (1) the transformation of the plant cell with a heterologous gene encoding an ABC-transporter or by (2) the overexpression of a homologous ABC-transporter which expressing is rate-limiting in the plant cell or (3) by the relocalization of a homologous or heterologous ABC-transporter from a normal membrane localization towards a vacuolar localization. The import of the intermediary product, or an amount produced thereof, into the vacuole reduces the negative feedback inhibition of the enzymatic reaction which occurs outside the vacuole and consequently enhances the production of the secondary metabolite in the plant cell.

In another embodiment, the current invention can be combined with other known methods to enhance the production and/or the secretion of secondary metabolites in plant cell cultures such as (1) by improvement of the plant cell culture conditions, (2) by the transformation of the plant cells with a transcription factor capable of upregulating genes involved in the pathway of secondary metabolite formation, (3) by the addition of specific elicitors to the plant cell culture, and 4) by the induction of organogenesis.

In another embodiment of the invention, DNA sequences encoding ABC-transporters are used to enhance the production of at least one secondary metabolite in plants comprising the transformation of the plants with an expression vector comprising an expression cassette further comprising a gene coding for an ABC-transporter.

By the term “to enhance the production” it is meant that the level of one or more metabolites may be enhanced by at least 20%, 30%, 40%, 50%, 60%, 70%, 80%, 90% or at least 100% relative to the untransformed plant which was used to transform with an expression vector comprising an expression cassette further comprising a gene coding for a transporter or an ABC-transporter. An enhanced production of a secondary metabolite can result in a detection of a higher level of secondary metabolites in the plant, for example in the vacuole. In another embodiment, the enhanced production of at least one secondary metabolite leads to an enhanced extracellular secretion of the secondary metabolite. In yet another embodiment, the same production of at least one secondary metabolite occurs in the transformed plant but an enhanced secretion of at least one secondary metabolite occurs by the transformed plant. Secondary metabolites can for example be efficiently produced by continuous secretion from the roots of hydroponically grown plants. This process of secretion is also been termed ‘rhizosecretion’.

The term “plant” as used herein refers to vascular plants (e.g., gymnosperms and angiosperms). The method comprises transforming a plant cell with an expression cassette of the present invention and regenerating such plant cell into a transgenic plant. Such plants can be propagated vegetatively or reproductively. The transforming step may be carried out by any suitable means, including by Agrobacterium-mediated transformation and non-Agrobacterium-mediated transformation, as discussed in detail below. Plants can be regenerated from the transformed cell (or cells) by techniques known to those skilled in the art. Where chimeric plants are produced by the process, plants in which all cells are transformed may be regenerated from chimeric plants having transformed germ cells, as is known in the art. Methods that can be used to transform plant cells or tissue with expression vectors of the present invention include both Agrobacterium and non-Agrobacterium vectors. Agrobacterium-mediated gene transfer exploits the natural ability of Agrobacterium tumefaciens to transfer DNA into plant chromosomes and is described in detail in Gheysen, G., Angenon, G. and Van Montagu, M. 1998. Agrobacterium-mediated plant transformation: a scientifically intriguing story with significant applications. In K. Lindsey (Ed.), Transgenic Plant Research. Harwood Academic Publishers, Amsterdam, pp. 1-33 and in Stafford, H. A. (2000) Botanical Review 66: 99-118. A second group of transformation methods is the non-Agrobacterium mediated transformation and these methods are known as direct gene transfer methods. An overview is brought by Barcelo, P. and Lazzeri, P. A. (1998) Direct gene transfer: chemical, electrical and physical methods. In K. Lindsey (Ed.), Transgenic Plant Research, Harwood Academic Publishers, Amsterdam, pp. 35-55. Hairy root cultures can be obtained by transformation with virulent strains of Agrobacterium rhizogenes, and they can produce high contents of secondary metabolites characteristic to the mother plant. Protocols used for establishing of hairy root cultures vary, as well as the susceptibility of plant species to infection by Agrobacterium (Toivunen L. (1993) Biotechnol. Prog. 9, 12; Vanhala L. et al. (1995) Plant Cell Rep. 14, 236). It is known that the Agrobacterium strain used for transformation has a great influence on root morphology and the degree of secondary metabolite accumulation in hairy root cultures. It is possible that by systematic clone selection e.g., via protoplasts, to find high yielding, stable, and from single cell derived-hairy root clones. This is possible because the hairy root cultures possess a great somaclonal variation. Another possibility of transformation is the use of viral vectors (Turpen T H (1999) Philos Trans R Soc Lond B Biol Sci 354(1383): 665-73).

Any plant tissue or plant cells capable of subsequent clonal propagation, whether by organogenesis or embryogenesis, may be transformed with an expression vector of the present invention. The term ‘organogenesis’ means a process by which shoots and roots are developed sequentially from meristematic centers; the term ‘embryogenesis’ means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include protoplasts, leaf disks, pollen, embryos, cotyledons, hypocotyls, megagametophytes, callus tissue, existing meristematic tissue (e.g., apical meristems, axillary buds, and root meristems), and induced meristem tissue (e.g., cotyledon meristem and hypocotyls meristem).

These plants may include, but are not limited to, plants or plant cells of agronomically important crops, such as tomato, tobacco, diverse herbs such as oregano, basilicum and mint. It may also be applied to plants that produce valuable compounds, e.g., useful as for instance pharmaceuticals, as ajmalicine, vinblastine, vincristine, ajmaline, reserpine, rescinnamine, camptothecine, ellipticine, quinine, and quinidien, taxol, morphine, scopolamine, atropine, cocaine, sanguinarine, codeine, genistein, daidzein, digoxin, colchicines, calystegins or as food additives such as anthocyanins, vanillin; including but not limited to the classes of compounds mentioned above. Examples of such plants include, but not limited to, Papaver spp., Rauvolfia spp., Taxus spp., Cinchona spp., Eschscholtzia californica, Camptotheca acuminata, Hyoscyamus spp., Berberis spp., Coptis spp., Datura spp., Atropa spp., Thalictrum spp., Peganum spp.

In another embodiment, the invention provides an isolated polypeptide selected from the groups consisting of (a) an isolated polypeptide encoded by a polynucleotide comprising the sequence of SEQ ID NO: 1 of the accompanying SEQUENCE LISTING, the contents of which are incorporated by this reference; (b) an isolated polypeptide comprising a polypeptide sequence having a least 83% identity to the polypeptide sequence of SEQ ID NO: 2; (c) fragments and variants of such polypeptides in (a) to (b) that induce or enhance the production or the secretion of at least one secondary metabolite in plants, plant cells, or extracellularly to the plant cells.

In another embodiment, the invention provides an isolated polynucleotide selected from the groups consisting of (a) an isolated polynucleotide comprising a polynucleotide sequence of SEQ ID NO: 1; (b) an isolated polynucleotide comprising a polynucleotide sequence having at least 91% identity to SEQ ID NO: 1; (c) fragments and variants of such polynucleotides in (a) to (b) that induce or enhance the production or the secretion of at least one secondary metabolite in plants, plant cells, or extracellularly to the plant cells.

As used herein, the words “polynucleotide” may be interpreted to mean the DNA and cDNA sequence as detailed by Yoshikai et al. (1990) Gene 87:257, with or without a promoter DNA sequence as described by Salbaum et al. (1988) EMBO J. 7(9):2807.

As used herein, “fragment” refers to a polypeptide or polynucleotide of at least about 9 amino acids or 27 base pairs, typically 50 to 75, or more amino acids or base pairs, wherein the polypeptide contains an amino acid core sequence. If desired, the fragment may be fused at either terminus to additional amino acids or base pairs, which may number from 1 to 20, typically 50 to 100, but up to 250 to 500 or more. A “functional fragment” means a polypeptide fragment possessing the biological property of that induce or enhance the production or the secretion of at least one secondary metabolite in plants, plant cells, or extracellularly to the plant cells. The terms ‘identical’ or percent ‘identity’ in the context of two or more nucleic acids or polypeptide sequences, refer to two or more sequences or subsequences that are the same or have a specified percentage of amino acid residues or nucleotides that are the same (i.e., 70% identity over a specified region), when compared and aligned for maximum correspondence over a comparison window, or designated region as measured using sequence comparison algorithms or by manual alignment and visual inspection. Preferably, the identity exists over a region that is at least about 25 amino acids or nucleotides in length, or more preferably over a region that is 50-100 amino acids or nucleotides or even more in length. Examples of useful algorithms are PILEUP (Higgins & Sharp, CABIOS 5:151 (1989), BLAST and BLAST 2.0 (Altschul et al. J. Mol. Biol. 215: 403 (1990). Software for performing BLAST analyses is publicly available through the National Center for Biotechnology Information (www/ncbi.nlm.nih.gov).

The invention is further explained with the aid of the following illustrative Examples:

### EXAMPLES

The recombinant DNA and molecular cloning techniques applied in the below examples are all standard methods well known in the art and are, for example, described by Sambrook et al. Molecular cloning: A laboratory manual (Cold Spring Harbor Laboratory Press, 2d ed. 1989). Methods for yeast culture and manipulation applied in the below examples are all standard methods well known in the art and are described, for example, in Guthrie and Fink Guide to yeast genetics and molecular biology, (Academic Press, Inc., New York, N.Y. 1991). Methods for tobacco cell culture and manipulation applied in the below examples are methods described in or derived from methods described in Nagata et al. (1992) Int. Rev. Cytol. 132, 1.

### Example 1

### Identification of Yeast Multidrug Resistance Transporters Specific for Tropane (Tas) and Nicotine-Type Alkaloids (NAs)

In the yeast Saccharomyces cerevisiae, a complex pleiotropic drug resistance (PDR) network of genes involved in multidrug resistance is composed of the transcriptional regulators Pdr1p and Pdr3p, which activate expression of the ATP-binding cassette (ABC) transporter-encoding genes PDR5, SNQ2, YOR1, as well as other not yet identified genes. To assess yeast sensitivity towards tropane alkaloids (Tas) and nicotine alkaloids (Nas) and identify yeast ABC transporters with specificity for TAs and NAs, we have screened isogenic yeast strains deleted of the ABC transporters YOR1, SNQ2, PDR5, PDR10, PDR11 or YCF1 for tolerance to the toxic compounds hyoscyamine, scopolamine and nicotine. The isogenic yeast strains derived from the US50-18C genotype were constructed and described in Decottignies et al. (J. Biol. Chem. (1998) 273, 12612). The yeast strains derived from the BY4741 genotype are obtained from the EUROSCARF collection (Frankfurt, Germany). All strains are listed in Table 1.

TABLE 1
Yeast strains used

| Strain | Genotype |
|---|---|
| US50-18C | Mata pdr 1-3 ura3 his1 |
| AD1 | US50-18C yor1::hisG |
| AD2 | US50-18C snq2::hisG |
| AD3 | US50-18C pdr5::hisG |
| AD4 | US50-18C pdr10::hisG |
| AD5 | US50-18C pdr11::hisG |
| BY4741 | Mata his3Δ1 leu2Δ0 met15Δ0 ura3Δ0 |
| Y02409 | BY4741 pdr5::kanMX4 |
| Y03951 | BY4741 snq2::kanMX4 |
| Y04069 | BY4741 ycf1::kanMX4 |
| Y05933 | BY4741 yor1::kanMX4 |

Alkaloid tolerance was assessed by controlling growth performance on rich medium (YPD) that contained different concentrations of TAs or NAs. To this end the different strains were grown to saturation (48 h) in liquid YPD. Cultures were diluted 10-, 100- and 1000-fold, and volumes of about 3 μl were dropped with a stainless steel replicator on YPD plates containing 2% Bacto Agar with the toxic compounds. Rich medium contains 1% yeast extract, 2% Bacto Peptone and 2% glucose. Filter-sterilized water solutions of hyoscyamine, scopolamine and nicotine were added after autoclaving. Growth was evaluated after two days incubation at 28° C. We observed that wild type yeast (i.e., not deleted for one of the ABC transporters) can tolerate hyoscyamine, scopolamine and nicotine to levels of 50 mM, 100 mM, and 15 mM respectively. Gradually increasing alkaloid levels in the medium caused growth retardation and was finally lethal. All isogenic strains except the pdr5 mutant strain showed identical alkaloid sensitivity. The above-mentioned alkaloid concentrations were lethal for the strain deleted for the PDR5 gene. This indicates that Pdr5p shows substrate specificity for TAs and NAs and is the only known ABC transporter involved in TA or NA transport in yeast cells. Previously other plant secondary metabolites such as indole alkaloids (e.g., vinblastine and vincristine), taxol and flavonoids were also shown to be substrates for Pdr5p mediated multidrug transport (Kolaczkowski et al. (1996) J. Biol. Chem. 271, 31543 and Kolaczkowski et al. (1998) Microb. Drug Resist. 4, 143).

### Example 2

### Assessment of Toxicity of Tas and Nas to Tobacco BY-2 Suspension Cultured Cells

Suspension cultured tobacco cells, Nicotiana tabacum L. cv Bright Yellow 2 were grown in the dark at 26° C. on a rotary shaker (130 rpm) in MSST, a modified Murashige-Skoog basal medium supplemented with 1.5 mM KH₂PO₄, 3 μM thiamine, 0.55 mM inositol, 87 mM sucrose and 1 μM 2,4D. Cells are subcultured every 7 days by transferring 0.5 ml into 50 ml of fresh medium in 250-ml flasks.

Toxicity of TAs and NAs to tobacco BY-2 cells was assessed in two ways. In the first method growth performance on MSST medium containing different concentrations of TAs or NAs was controlled. To this end a fresh BY-2 cell culture was started and after 3 days culture volumes of about 300 μl were dropped on MSST plates containing 0.65% Bacto Agar and the toxic alkaloids. Filter-sterilized water solutions of hyoscyamine and nicotine were added after autoclaving. Growth was evaluated after 15 days incubation at 26° C. Wild type BY-2 cells (i.e., not transgenic) can tolerate hyoscyamine and nicotine without severe growth problems to levels of 30 mM and 3 mM respectively. A gradually increasing alkaloid level in the medium caused growth retardation and finally was lethal. In the second method toxicity was evaluated by measuring cell death after incubation in the presence of increasing levels of alkaloids. Cell death was scored by the Evans blue method (Turner and Novacky (1974) Phytopathol. 64, 885). To this end a fresh BY-2 cell culture was started and after 3 days 5 ml of this culture was transferred to one well of a 6-well plate (Falcon 353046). 1 ml of fresh MSST was added and the desired toxic compound in a volume of 650 μl in 0.1M potassium phosphate buffer at pH 5.8. Cells were then further incubated on the rotary shaker and 1-ml samples were taken after 0, 6 and 24 hours. We spun the cells down at 6000 rpm for 3 minutes, removed the supernatant, added 1 ml of 0.1% Evans blue in MSST medium and incubated for 15 minutes at room temperature on a rotary wheel. Afterwards we spun the cells down again and washed 5 times with fresh MSST medium till all the blue color was gone from the supernatant. Dye bound to dead cells was solubilized by incubation in 1 ml of 50% methanol, 1% SDS for 30 minutes at 50° C. We spun the cells down again (now at 14000 rpm for three minutes) and quantified cell death by measuring OD₆₀₀ of the supernatant. Cell death is expressed as fold increase in Evans blue staining compared to the control cells. In this assay tobacco BY-2 cells are found sensitive to all the compounds tested. Hyoscyamine and nicotine cause the death of all suspension cultured tobacco cells within 24 hours of incubation at levels of 50 mM and 20 mM respectively. This indicates that the metabolites that plants produce inside the cells can be toxic for themselves and also that this toxicity can result in slow growth of plant cells producing secondary metabolites. Furthermore these results provided us with useful assay systems for evaluating the activity of ABC transporters from different organisms such as yeast, plants and animals in tobacco cell suspension cultures.

### Example 3

### Expression of PDR5 in Tobacco BY-2 Suspension Cultured Cells

3.1 Cloning of PDR5

The PDR5 gene was cloned by the PCR method with the PfuI polymerase. To this end oligonucleotides were designed with 5′-terminal attB sequences that amplify the entire open reading frame of the PDR5 gene (4536 nt) as a PCR product that is an efficient substrate for recombination with the Gateway™ system (Invitrogen). Gateway technology provides an alternative rapid method for cloning a sequence into a multiple expression system. The advantage of the Gateway cloning is that fragments present as Entry clones can be subcloned into different Destination vectors in a short time. This technology was used to construct a set of versatile vectors for Agrobacterium-based plant transformation. Our intention was to develop vectors for wide range plant gene analysis. The Gateway-compatible binary vector pPZP200 is the backbone of our constructs (Hajdukiewicz et al. Plant Molecular Biology 25, 989-994, 1994). This binary vector is relatively small in size, contains two origins of replication in E. coli or in Agrobacterium and posses streptomycin and/or spectinomycin for plasmid selection. Three plant selectable marker genes; kanamycin, hygromycin and bar (most frequently used markers in plant transformation) have been used for all constructs. All selectable markers are in a cassette containing nos (nopaline synthase) promoter and nos terminator. These genes were cloned toward the left border of the T-DNA. For construction of all Gateway clones we have used the rfA conversion cassette.

The oligonucleotides used for PDR5 gene cloning, are

(SEQ ID NO: 3)

5′-AAAAGCAGGCTACCATGCCCGAGGCCAAGCTTAACAATA-3′

as the forward primer and

(SEQ ID NO: 4)

5′-AGAAAGCTGGGTCCATCTTGGTAAGTTTCTTTTCTTAACC-3′

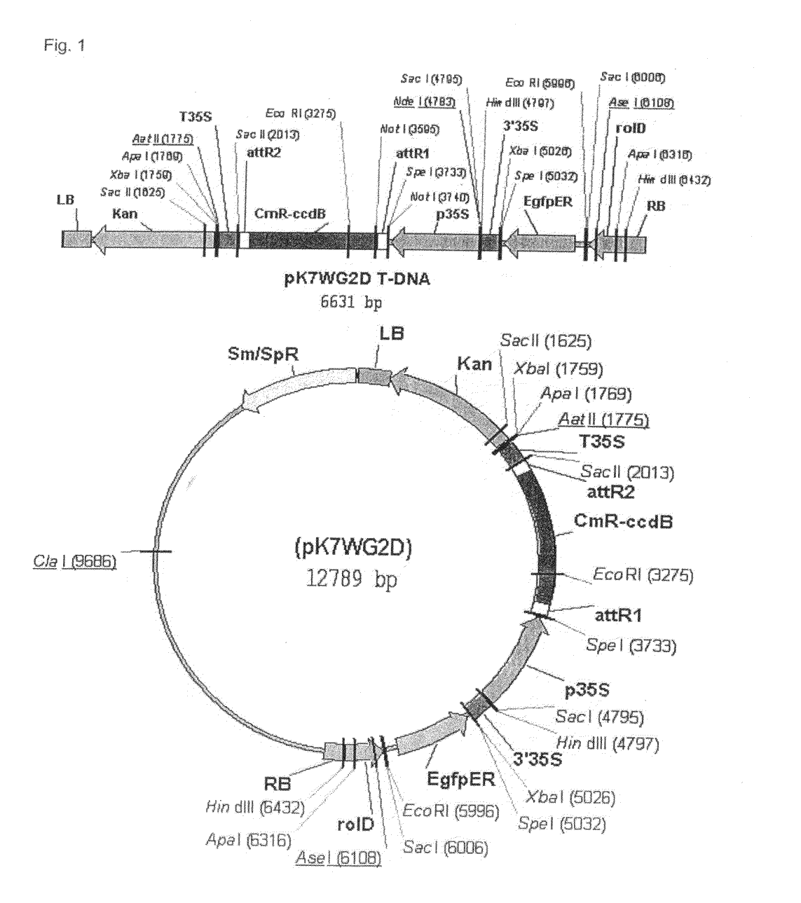

as the reverse primer, respectively. As a template, genomic DNA prepared from the yeast strains US50-18C or W303 was used. First the PCR fragments were introduced in the Donor Vector pDONR201 (Invitrogen) via the BP reaction to generate the Entry Clone. Then the PDR5 gene was transferred to the Destination Vector pK7WGD2 (FIG. 1) via the LR reaction, where the gene is under control of the CaMV 35S promoter. The T-DNA of the pK7WGD2 binary vector also bears the kanamycin resistance gene (NPTII) under the control of the pnos promoter as selectable marker for plant transformation and the gene encoding the green fluorescent protein (GFP) under the control of the prolD promoter for visual selection of transgenic plant cell lines. The resulting binary plasmids were designated pK7WGD2-ScPDR5-US50 or pK7WGD2-ScPDR5-W303 depending on the yeast genotype from which the gene is isolated. Also the GUS gene was introduced in the pK7WGD2 vector and the resulting binary vector pK7WGD2-GUS served as a control for the experiments described in the examples below.

3.2 Transformation of Tobacco BY-2 Suspension Cultured Cells

Plant cell transformations were carried out by applying the ternary vector system (van der Fits et al. (2000) Plant Mol. Biol. 43, 495). The plasmid pBBR1MCS-5.virGN54D is used as a ternary vector. The binary plasmid was introduced into Agrobacterium tumefaciens strain LBA4404 already bearing the ternary plasmid by electro-transformation.

Agrobacterium tumefaciens strains were grown for three days at 28° C. on solid LC medium containing 20 μg/ml rifampicin, 40 μg/ml geneticin, 100 μg/ml spectinomycin and 300 μg/ml streptomycin. LC medium contains 1% Bacto Trypton, 0.5% Bacto yeast extract and 0.8% NaCl. From these bacteria a 5-ml liquid culture was grown in LC medium for 48 hours. N. tabacum BY-2 cells were grown in MSST medium as described in example 2. For transformation 3 days old cell cultures were used. For cocultivation 4 ml of BY-2 cells was transferred to the corner of a Petri dish (ø 80 mm) and 300 μl of the A. tumefaciens culture was added. Dishes were taped with respiratory tape and incubated for 3 days at 26° C. in the dark. After 3 days the cocultivation mixture was transferred into 20 ml of fresh MSST medium 50 μg/ml kanamycin-B, 500 μg/ml carbenicillin and 250 μg/ml vancomycin in 100-ml flasks and further incubated as described in example 2. After one week 4 ml of this cell suspension culture was subcultured in 40 ml of fresh MSST medium with 10 μg/ml of the kanamycin analogue G-418 (geneticin), 500 μg/ml carbenicilin and 250 μg/ml vancomycin and grown further till it reached maximal density (similar to stationary, 1-week-old culture) which took two to three weeks, depending on the efficiency of the transformation event. After two additional 1 ml transfer cycles in medium containing 50 μg/ml kanamycin-B, 500 μg/ml carbenicilin and 250 μg/ml vancomycin cells were further propagated in an antibiotic-free MSST medium as described in example 2. Elimination of agobacteria was verified and efficient transgene expression was scored in vivo by observing GFP fluorescence with a fluorescence microscope equipped with HQ-GFP band-pass filters for an excitation at 470 and emission at 525 nm.

3.3 Effect of Heterologous PDR5Expression in BY-2 Suspension Cultured Cells on Alkaloid Tolerance

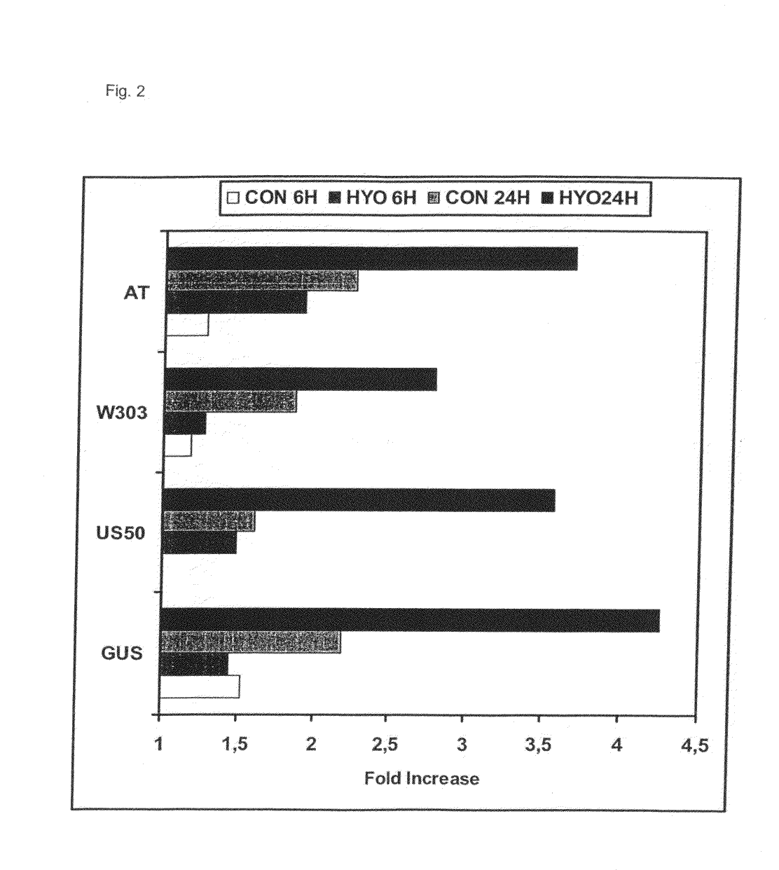

In recombinant BY-2 cells transformed with the PDR5 expression cassettes (from both yeast genotypes), correct PDR5 expression is tested by northern blot analysis using a PDR5 specific DNA probe and by western blot analysis using a rabbit polyclonal anti-Pdr5p antibody (Decottignies et al. (1999) J. Biol. Chem. 274, 37139). In both lines PDR5 is efficiently expressed both on the RNA and protein level. Fractionation also shows that the Pdr5 protein is correctly targeted to the plasma membrane. Tolerance of the transformed BY-2 suspension cultures to hyoscyamine and nicotine was assessed by the two assays described in example 2. As can be deduced from the growth performance assay, BY-2 cell lines expressing the different yeast Pdr5 transporters displayed to varying extents an increased tolerance to both alkaloids as compared to the control GUS-expressing lines. Lines expressing the PDR5 transporter from yeast genotype W303 showed the highest alkaloid tolerance, in particular towards hyoscyamine. In the cell death experiment hyoscyamine was added to a final concentration of 30 mM. Transgene BY-2 cells expressing the Pdr5p from yeast strain W303 again showed the highest tolerance to this tropane alkaloid (FIG. 2). Fold increase in cell death lowered with ca. 35% in the W303 lines whereas US50 lines had a 15% decrease in hyoscyamine induced cell death.

3.4 Effect of Heterologous PDR5 Expression in BY-2 Suspension Cultured Cells on Nicotinic Alkaloid Production

For the analysis of nicotinic alkaloid accumulation, 6-day old recombinant BY-2 cell cultures (BY-2 transformed with pK7WGD2-ScPDR5-US50 or pK7WGD2-ScPDR5-W303 or pK7WGD2-GUS) were washed and diluted ten-fold with fresh hormone free MSST medium. After a recuperation period of 12 hours, the cultures were treated with methyl jasmonate (MeJA). MeJA was dissolved in dimethyl sulfoxide (DMSO) and added to the culture medium at a final concentration of 50 μM. As a control, cells treated with an equivalent amount of DMSO were included. For alkaloid analysis, three replicate shake flasks with a volume of 20 ml were processed. After vacuum-filtering through Miracloth, cells and medium were separated from each other for intracellular and extracellular alkaloid analysis respectively. The filtered cell mass was transferred to a test tube, frozen and lyophilized (50 mbar, approx. 48 hours). Lyophilized cell samples were extracted for GC-MS analysis by a modified method described by Furuya et al. (1971, Phytochemistry, 10, 1529). Cells were weighed and 25 μg 5-α-cholestane was added as internal standard. The samples are made alkaline with ammonia (10% (v/v), 1 ml) and water (2 ml) is added. Alkaloids were extracted by vortexing with 2 ml of dichloromethane. After 30 minutes, the samples were centrifuged (2000 rpm, 10 min) and the lower organic layer was separated and transferred into glass vials. After evaporation to dryness 25 μl of dichloromethane was added and the samples were silylated with N-methyl-N-(trimethylsilyl)trifluoroacetamide (Pierce, Rockford, Md., US) for 20 min at 120° C. prior to GC-MS analysis. For alkaloid determination in the medium, 20 ml of the filtered medium was made alkaline with ammonia (10% v/v) to reach pH 9. Internal standards were added (5-α-cholestane and cotinine). Subsequently this solution was extracted twice with dichloromethane (1:1) and evaporated to dryness. The column was rinsed twice with 1 ml of dichloromethane and the extract was transferred into glass vials. We further proceeded as described above for the cell extract.

TABLE 2
Alkaloid accumulation in transformed BY-2 cells^{a}

| | | |
|---|---|---|
| BY-2 Strain | Nicotine^{b} | Anatabine^{b} |

| medium | Medium | Cells | Medium | Cells % | in |
|---|---|---|---|---|---|
| GUS | 0 | 2.00 | 0.18 | 157 | 0.1 |
| ScPDR5- | 0 | 0.88 | 7.40 | 207 | 3.6 |
| US50 | | | | | |
| ScPDR5- | 0 | 2.03 | 5.12 | 74 | 6.9 |
| W303 | | | | | |

^{a}Measured 72 hours after elicitation with 50 μM methyl jasmonate. Results are the mean of three independent experiments
^{b}Indicated in μg/flask, with 20-ml BY-2 culture per flask

In jasmonate elicited BY-2 cells, the alkaloids detected after 72 hours are nicotine, anabasine, anatabine, and anatalline. No alkaloids are detected in DMSO-treated samples, neither in the cells nor in the medium. The results for nicotine and anatabine are shown in Table 2. Of all alkaloids that are produced by elicited BY-2 cells only anatabine is found in the medium. Although only trace amounts of anatabine can be detected extracellularly, comparison of anatabine levels in the different BY-2 cell lines after 72 hours of MeJA treatment clearly shows an enhancement of anatabine export in cell lines transformed with the PDR5 genes.

### Example 4

### Expression of Vacuole Targeted PDR5 in Tobacco BY-2 Suspension Cultured Cells

4.1 Construction and Cloning of Recombinant PDR5

To target the yeast PDR5 protein to plant vacuolar membranes, two strategies are followed. In the first, the N-terminal signal peptide and pro-peptide from sweet potato (MKAFTLALFLALSLYLLPNPAHSRFNPIRLPTTHEPA (SEQ ID NO:5), Matsuoka and Nakamura (1991) Proc. Natl. Acad. Sci. USA 88, 834) are fused at the N-terminus of the Pdr5 protein. The resulting recombinant open reading frame is designated ScNVacPDR5. In the second approach the C-terminal amino acids of the tobacco chitinase A (DLLGNGLLVDTM (SEQ ID NO:6), Neuhaus et al. (1991) Proc. Natl. Acad. Sci. USA 88, 10362) are added at the C-terminus of the Pdr5 protein. The resulting recombinant open reading frame is designated ScPDR5CVac. Both recombinant genes are put under the control of the CaMV35S promoter and cloned in the binary vector bearing the HYG and GFP genes as described in Example 3.1. The resulting binary plasmids are designated pH-ScNVacPDR5-GFP and pH-ScPDR5CVac-GFP, respectively.

4.2 Effect of Recombinant PDR5 Expression in BY-2 Suspension Cultured Cells on Alkaloid Tolerance and Nicotine Production

BY-2 suspension cultured cells are transformed as described in Example 3.2 and 5 transgene calli of both ScNVacPDR5 or ScPDR5CVac transformed cells and highly expressing GFP are selected as described in Example 3.3. Control of expression of recombinant PDR5 is performed as described in Example 3.3 by northern and western blot analysis. Fractionation shows that in both types of transgene lines (NVac or CVac) the Pdr5 protein is targeted to the vacuolar membrane.

To assess tolerance to nicotine and hyoscyamine in transgenic cell lines the same assays as described in Example 3.3 are used here to evaluate the functionality of vacuole targeted Pdr5p. The effect of the vacuolar expression of PDR5 on nicotine production in BY-2 cells is evaluated as described in Example 3.4.

### Example 5

### Expression of Plant PDR Orthologues in Tobacco BY-2 Suspension Cultured Cells

5.1. Cloning of AtPDR1

The ABC protein super-family is the largest protein family known and most are membrane proteins active in the transport of a broad range of substances across the membranes. Also in Arabidopsis this superfamily is large and diverse (129 ORFs) and a complete inventory has been described by Sanchez-Fernandez et al. (J. Biol. Chem. (2001), 276, 30231). One of the subfamilies of full-length ABC transporters in Arabidopsis consists of the PDRs (13 ORFs) of which yeast PDR5 is the prototype. At least eight of the PDR5-like ORFs in Arabidopsis are transcriptionally active and have been isolated as ESTs (Sanchez-Fernandez et al. (2001), J. Biol. Chem., 276, 30231). Amongst these is one of the closest Arabidopsis PDR5-orthologues, namely the AtPDRJ gene (At3g16340) (SEQ ID NO: 16). A cDNA clone of the AtPDRJ gene (SEQ ID NO: 16) is isolated as described for the yeast PDR5 gene in Example 3.

To this end, the following oligonucleotides were designed:

(SEQ ID NO: 7)

5′-AAAAAGCAGGCTACCATGGAGACGTTATCGAGAA-3′

as the forward primer and

(SEQ ID NO: 8)

5′-AGAAAGCTGGGTCTATCGTTGTTGGAAGTTGAGC-3′

as the reverse primer, respectively. As a template we used cDNA prepared from Arabidopsis hypocotyls.

5.2 Cloning of HmPDR1

The biosynthesis of tropane alkaloids such as hyoscyamine and scopolamine in plants of the Solanaceae is very tissue-specific and occurs only in the roots. Later on, the alkaloids are transported to the aerial parts, especially the leaves, where they are finally accumulated. In hairy roots, however, this translocation cannot occur and part of the produced alkaloids is released in the medium. This release can be stimulated by the addition of millimolar amounts of CdCl₂ to the medium (Furze et al. (1991) Plant Cell Rep. 10, 111 and Pitta-Alvarez et al. (2000) Enzyme. Microb. Technol. 26, 252). This indicates the existence of active detoxifying mechanisms against cadmium in which also the tropane alkaloids would be involved. We applied this knowledge to isolate an alkaloid specific PDR-like gene from Hyoscyamus muticus hairy roots.

A cDNA clone of a PDR-like gene is isolated from H. muticus and is designated HmPDR1. To this end total RNA was prepared from hairy roots of the H. muticus KB7 line (Jouhikainen et al. (1999) Planta 208, 545) treated for 30 hours with 1 mM CdCl₂ and was reverse transcribed with the Superscript RTII reverse transcriptase. A nested PCR was subsequently carried out with the Taq DNA polymerase using the DNA-RNA hybrid as the template and two sets of degenerate primers designed from highly conserved amino acid sequences in the nucleotide binding folds of known yeast and plant PDR proteins (see, Table 3). This PCR yields two fragments derived from the two nucleotide-binding folds which are naturally present in the general tandem repeat structure of ABC proteins. Using specific primers and RT-PCR, 5′RACE and 3′RACE techniques we cloned a full-length cDNA clone, which is designated HmPDR1. The nucleotide sequence of the HmPDR1 cDNA clone is depicted in SEQ ID NO: 1, the amino acid sequence of the HmPDR1 protein is depicted in SEQ ID NO: 2.

TABLE 3
Degenerate primers used for HmPDR1 cDNA cloning

| Primer | Sequence |
|---|---|
| ALGG39 | 5′-CCIRGYKCIGGIAARACNAC-3′ |
| | (SEQ ID NO: 10) |
| ALGG40 | 5′-ACICKYTTYTTYTGNCCNCC-3′ |
| | (SEQ ID NO: 11) |
| ALGG41 | 5′-TCNARNCC-3′ |
| | (SEQ ID NO: 12) |
| ALGG42 | 5′-GGIGTIYTIACIGCNYTNATGGG-3′ |
| | (SEQ ID NO: 13) |
| ALGG43 | 5′-TCNARCATCCAIGTIGCNGGRTT-3′ |
| | (SEQ ID NO: 14) |
| ALGG44 | 5′-CKCCARTA-3′ |
| | (SEQ ID NO: 15) |

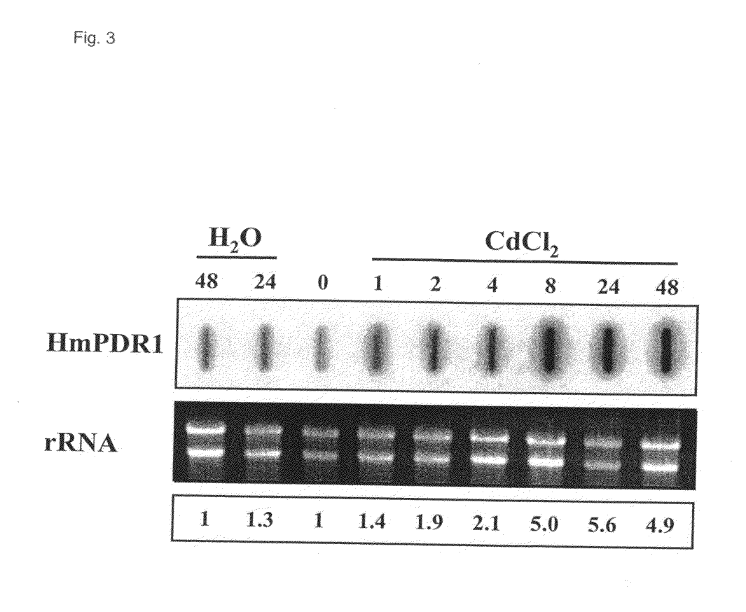

To confirm the postulated relationship between the expression of ABC transporter genes and the CdCl₂ induced release of alkaloids we performed an expression analysis of the HmPDR1 gene in CdCl₂ treated Hyoscyamus hairy roots (FIG. 3). Quantitative RT-PCR clearly showed that HmPDR1 is upregulated by CdCl₂ elicitation.

5.3 Effect of Heterologous AtPDR1 Expression in Yeast Cells on Alkaloid Tolerance

The AtPDR1 gene (SEQ ID NO: 16) was subcloned in a yeast expression vector (YCp50) between the 5′ and 3′ regulatory sequences of the yeast PDR5 gene. This plasmid was then introduced in the yeast AD3 strain (the pdr5 mutant, see Example 1). To analyze the substrate specificity of this plant PDR gene we controlled growth performance of the transformed yeast strains on YPD plates containing the different TAs and NAs as described in example 1. We have shown that the PDR1 gene (SEQ ID NO: 16) of A. thaliana was able to restore the growth of the pdr5 mutant strain on hyoscyamine and nicotine

5.4 Effect of Heterologous AtPDR1 Expression in BY-2 Suspension Cultured Cells On Alkaloid Tolerance

The AtPDR1 gene (SEQ ID NO: 16) was transferred to the binary vector pK7WGD2 as described in Example 3.1. BY-2 suspension cultured cells were transformed as described in example 3.2. Control of expression of AtPDR1 is performed by northern blot analysis using a specific DNA probe. To assess tolerance to nicotine and hyoscyamine in transgenic cell lines the same assays as described in Example 3.3 were performed in order to evaluate the functionality of AtPDR1p (SEQ ID NO: 17). Transgenic BY-2 cells showed enhanced tolerance to alkaloids as compared to the control GUS expressing line. However, not to the extent of the ScPDR5-W303 expressing line but comparable to the tolerance levels obtained in the ScPDR5-US50 line.

5.5 Effect of AtPDR1 Expression in BY-2 Suspension Cultured Cells on Nicotinic Alkaloid Production

For the analysis of nicotinic alkaloid accumulation, 6-day old recombinant BY-2 cell cultures (pK7WGD2-AtPDR1 en pK7WGD2-GUS) are washed and diluted ten-fold with fresh hormone free MSST medium. After a recuperation period of 12 hours, the cells are treated with methyl jasmonate (MeJA). MeJA is dissolved in dimethyl sulfoxide (DMSO) and added to the culture medium at a final concentration of 50 μM. As a control, cells treated with an equivalent amount of DMSO are included. For alkaloid analysis, the same process is followed as in Example 3.4.

What is claimed is:

1. A method of inducing or enhancing production of at least one secondary metabolite by plant cells, said method comprising:
transforming plant cells with an expression vector comprising an expression cassette comprising a gene encoding an ABC-transporter;
wherein said ABC-transporter comprises a Walker A box, a Walker B box, and a Nucleotide Binding Fold;
wherein said ABC-transporter functions to transport at least one secondary metabolite in plant cells;
selecting transformed plant cells having an induced or enhanced production of at least one secondary metabolite; and
propagating such selected transformed plant cells.

2. The method according to claim 1, wherein said gene encoding an ABC transporter comprises a polynucleotide sequence having at least 91% identity to the polynucleotide sequence of SEQ ID NO:17.

3. The method according to claim 1 wherein the secondary metabolites are alkaloids.

4. The method according to claim 1 wherein the ABC-transporters are of plant, fungal, or mammalian origin.

5. The method according to claim 1 wherein the induction or enhancement of the production of at least one secondary metabolite by plant cells results from enhancing the transport of said secondary metabolite into a vacuole.

6. The method according to claim 5 wherein the secondary metabolites are alkaloids.

7. The method according to claim 5 wherein the ABC-transporters are of plant, fungal, or mammalian origin.

8. The method according to claim 1 wherein the induction or enhancement of the production of at least one secondary metabolite by plant cells results from enhancing the transport of said secondary metabolite to the extracellular space of the plant cell.

9. The method according to claim 5 wherein the secondary metabolites are alkaloids.

10. The method according to claim 5 wherein the ABC-transporters are of plant, fungal, or mammalian origin.

11. A method of stimulating the production of secondary metabolites by plants, the method comprising:
transforming said plants with an expression vector comprising an expression cassette comprising a gene encoding an ABC-transporter;
wherein said ABC-transporter comprises a Walker A box, a Walker B box, and a Nucleotide Binding Fold; and
wherein said ABC-transporter functions to transport at least one secondary metabolite in plant cells;
selecting transformed plants based upon enhanced production of secondary metabolites; and
propagating such selected transformed plants.

12. The method according to claim 11, wherein said gene encoding an ABC transporter comprises a polynucleotide sequence having at least 91% identity to the polynucleotide sequence of SEQ ID NO:17.

13. The method according to claim 11 wherein the secondary metabolites are alkaloids.

14. The method according to claim 11 wherein the ABC-transporters are of plant, fungal, or mammalian origin.

15. A transgenic plant cell culture displaying an enhanced production of at least one secondary metabolite, wherein said transgenic plant cell is transformed with an expression vector comprising an expression cassette comprising a gene encoding an ABC-transporter;
wherein said ABC-transporter comprises a Walker A box, a Walker B box, and a Nucleotide Binding Fold; and
wherein said ABC-transporter functions to transport at least one secondary metabolite in plant cells.

16. The method according to claim 15, wherein said gene encoding an ABC transporter comprises a polynucleotide sequence having at least 91% identity to the polynucleotide sequence of SEQ ID NO:17.

17. The transgenic plant cell culture of claim 15 further characterized in having
(1) an increased vacuolar localization of said at least one secondary metabolite, or
(2) a secretion or an increased secretion of said at least one secondary metabolite.

18. A transgenic plant material selected from the group consisting of a plant, plant cells, plant seeds and plant progeny, said transgenic plant material capable of an enhanced production of at least one secondary metabolite, said transgenic plant material transformed with an expression vector comprising an expression cassette comprising a gene encoding an ABC-transporter;
wherein said ABC-transporter comprises a Walker A box, a Walker B box, and a Nucleotide Binding Fold; and
wherein said ABC-transporter functions to transport at least one secondary metabolite in plant cells.

19. The transgenic plant material of claim 18, wherein said gene encoding an ABC transporter comprises a polynucleotide sequence having at least 91% identity to the polynucleotide sequence of SEQ ID NO:17.

20. The transgenic plant material of claim 18 further characterized in having an increased vacuolar localization of said at least one secondary metabolite.

21. An isolated polynucleotide sequence comprising a sequence having at least 91% identity to the polynucleotide sequence of SEQ ID NO:17;
wherein the isolated polynucleotide sequence induces or enhances production of at least one secondary metabolite in plants.

22. A process for producing a plant cell exhibiting an enhanced production of at least one secondary metabolite, said process comprising:
transforming a plant cell with an expression cassette comprising a gene encoding an ABC-transporter;
wherein said ABC-transporter comprises a Walker A box, a Walker B box, and a Nucleotide Binding Fold;
wherein said ABC-transporter functions to transport at least one secondary metabolite in plant cells; and
selecting transformed plant cells exhibiting enhanced transport of said at least one secondary metabolite into a vacuole.

23. The process according to claim 22, wherein said gene encoding an ABC transporter comprises a polynucleotide sequence having at least 91% identity to the polynucleotide sequence of SEQ ID NO:17.

24. A plant cell produced by the process of claim 22.

25. A transgenic plant including the plant cell of claim 24.

26. An isolated polynucleotide useful for producing a plant cell exhibiting an enhanced production of at least one secondary metabolite, said isolated polynucleotide comprising:
a first sequence of nucleotide bases constituting a means for inducing or enhancing production of at least one secondary metabolite in plants or plant cells, and
a second sequence of nucleotides bases, operatively positioned with respect to said first sequence, constituting a means for promoting expression of said first sequence.

27. The isolated polynucleotide sequence of claim 26, wherein the isolated polynucleotide sequence comprises a polynucleotide sequence having at least 91% identity to the polynucleotide sequence of SEQ ID NO:17.

28. A method of inducing or enhancing production or cellular secretion of at least one endogenous secondary metabolite by a plant cell, the method comprising:
transforming the plant cell with an expression vector comprising an expression cassette comprising a gene encoding an ABC-transporter, wherein said ABC-transporter comprises a Walker A box, a Walker B box, and a Nucleotide Binding Fold, and functions to transport at least one secondary metabolite in plant cells;
wherein the secondary metabolite is an endogenous metabolic product of the plant cell, and is transported from the cell to the extracellular space;
wherein the amount of secondary metabolite recoverable from the cell is increased;
selecting a transformed plant cell having an induced or enhanced production of at least one secondary metabolite; and
propagating such selected transformed plant cell

29. The method according to claim 28, wherein said gene encoding an ABC transporter comprises a polynucleotide sequence having at least 91% identity to the polynucleotide sequence of SEQ ID NO:17.

30. The method according to claim 28 wherein the secondary metabolite is an alkaloid.

31. The method according to claim 28 wherein the ABC-transporter is of plant, fungal, or mammalian origin.
